(19) United States
(12) Patent Application Publication
(10) Pub. No.: US 2025/0134399 A1
(43) Pub. Date: May 1, 2025

(54) BLOOD PRESSURE RECORDING DEVICE AND BLOOD PRESSURE RECORDING METHOD

(71) Applicant: OMRON HEALTHCARE CO., LTD., Muko-shi (JP)

(72) Inventors: Yukiya SAWANOI, Kyoto (JP); Nobuki YAKURA, Kyoto (JP); Shingo YAMASHITA, Kyoto (JP)

(73) Assignee: OMRON HEALTHCARE CO., LTD., Muko-shi (JP)

(21) Appl. No.: 19/008,836

(22) Filed: Jan. 3, 2025

Related U.S. Application Data
(63) Continuation of application No. PCT/JP2023/036745, filed on Oct. 10, 2023.

(30) Foreign Application Priority Data
Feb. 17, 2023 (JP) 2023-023397

(51) Int. Cl. A61B 5/022; A61B 5/00; A61B 5/021

(52) U.S. Cl. CPC A61B 5/02208; A61B 5/02108; A61B 5/02141; A61B 5/02225; A61B 5/742; A61B 5/7475; A61B 2560/0462; A61B 2562/0204; A61B 2562/0247

(57) ABSTRACT

In the depressurization process, a memory is caused to sequentially store, as a data pair, a time at which the waveform feature point of the pulse beat was generated and the cuff pressure at the time, in association with each other. The cuff pressure is displayed on an indicator. In the depressurization process, an observer who listens to a Korotkoff sound with a stethoscope while viewing the cuff pressure displayed on the indicator operates an operation unit, and inputs a timing signal indicating a time at which the observer judged that generation of the Korotkoff sound had stopped generating. A data pair corresponding to one pulse beat before a data pair corresponding to the time is specified, and a cuff pressure included in the specified data pair is recorded as a corrected diastolic blood pressure value.

### CROSS-REFERENCE TO RELATED APPLICATIONS

This is a continuation application of International Application No. PCT/JP2023/036745, with an International filing date of Oct. 10, 2023, which claims priority of Japanese Patent Application No. 2023-023397 filed on Feb. 17, 2023, the entire content of which is hereby incorporated by reference.

### TECHNICAL FIELD

This invention relates to a blood pressure recording device, and more particularly to a blood pressure recording device that measures and records a blood pressure based on a Korotkoff sound generated by an artery through a measurement target site. This invention also relates to a blood pressure recording method for measuring and recording a blood pressure using such a blood pressure recording device.

### BACKGROUND ART

As this type of blood pressure recording device, for example, as disclosed in Non-Patent Document 1 (“UM-102/UM-102B Mercury-Free Sphygmomanometer Instruction Manual”, A&D Company, Limited, Internet <URL:https://www.aandd.co.jp/products/medical/equipment/mesphygmomanometer/um102/>), a manual electronic sphygmomanometer including a pressure indicator that electronically displays, as a bar graph along a scale, a pressure (cuff pressure) of a cuff worn on an upper arm of a subject is known in place of a traditional mercury sphygmomanometer. This electronic sphygmomanometer (conventional example) includes a HOLD button, and when an observer (a medical doctor, a nurse, and the like) presses the HOLD button upon judging a maximum blood pressure or a minimum blood pressure by auscultatory method in a depressurization process (during blood pressure measurement), a pressure value at that time is displayed in a number and held (substantially recorded) (hold function).

### SUMMARY OF THE INVENTION

As described in Non-Patent Document 2 (George S. Stergiou et al, “Validation of the A&D UM-101 professional hybrid device for office blood pressure measurement according to the International Protocol”, Blood Pressure Monitoring 2008, Vol. 13, No. 1, pp 37-42), in the electronic sphygmomanometer, when the HOLD button (mark button) is used, there is a problem that the blood pressure is systematically evaluated lower than the actual blood pressure, and the validation criteria cannot be satisfied. The same document includes consideration that the reason is due to the reaction time required for the observer to press the HOLD button (mark button).

Here, when the present inventor repeated a blood pressure measurement experiment using the hold function of the electronic sphygmomanometer, it was found that the tendency that the blood pressure is evaluated to be lower than the actual blood pressure appears more remarkably in the minimum blood pressure than the maximum blood pressure. Specifically, using the electronic sphygmomanometer, the present inventor simultaneously performed a blood pressure measurement by a normal auscultatory method and a blood pressure measurement using the hold function. In the blood pressure measurement by the normal auscultatory method, the observer read the maximum blood pressure and the minimum blood pressure based on a Korotkoff sound heard with a stethoscope while viewing the cuff pressure displayed in real time as a bar graph on the pressure indicator (this read value is called an “auscultatory value”). On the other hand, in the blood pressure measurement using the hold function, the observer pressed the HOLD button when judging the maximum blood pressure or the minimum blood pressure based on a Korotkoff sound heard with a stethoscope while viewing the cuff pressure displayed in real time as a bar graph on the pressure indicator, and recorded the cuff pressure at that time (this recorded value is called a “HOLD value”). Then, a difference (HOLD value−auscultatory value) between the obtained HOLD value and the auscultatory value was obtained for each of 60 measurements. FIG. 22A shows a scatter diagram where the auscultatory value is represented on the horizontal axis and the difference (HOLD value−auscultatory value) is represented on the vertical axis, for the obtained maximum blood pressure. FIG. 22B shows a scatter diagram where the auscultatory value is represented on the horizontal axis and the difference (HOLD value−auscultatory value) is represented on the vertical axis, for the obtained minimum blood pressure. As seen from these figures, the mean value of the differences (HOLD value−auscultatory value) for the maximum blood pressure was −1.5 mmHg. On the other hand, the mean value of the differences (HOLD value−auscultatory value) for the minimum blood pressure was −3.2 mmHg. In this manner, when the hold function of the electronic sphygmomanometer was used, the tendency that the blood pressure is evaluated to be lower than the actual blood pressure (auscultatory value) appeared more remarkably in the minimum blood pressure.

The reason for this is analyzed as follows. The upper part of FIG. 21 shows a transition of an intra-arterial pressure Pa and a change in cuff pressure Pc with the lapse of time. The intra-arterial pressure Pa shows, between a minimum blood pressure DIA and a maximum blood pressure SYS, a mountain-like waveform that rises, shows a peak and falls, for each pulse beat of the pulse wave. By an air bulb, the cuff pressure Pc is temporarily pressurized to Pcmax higher than an expected maximum blood pressure by about 30 mmHg, and subsequently depressurized at a depressurization rate of typically 2 to 3 mmHg/sec. Then, when the cuff pressure Pc falls below the maximum blood pressure SYS, as shown in the lower part of FIG. 21, a Korotkoff sound Kc starts generating for each pulse beat, and when the cuff pressure Pc further falls below the minimum blood pressure DIA, the Korotkoff sound Kc stops generating. Here, for the maximum blood pressure SYS, the observer presses the HOLD button at a time when he/she can judge that the Korotkoff sound Kc has started generating, that is, at a time t1 when he/she hears a first Korotkoff sound Kcf from a silent state (note that for the sake of simplicity, the lower part of FIG. 21 omits illustration of reaction time of the observer). Therefore, the observer can press the HOLD button at a relatively correct time. On the other hand, for the minimum blood pressure DIA, the observer presses the HOLD button at a time when he/she no longer hears the Korotkoff sound Kc from a state where he/she continued to hear the Korotkoff sound Kc for each pulse beat, specifically, at a time t2 when he/she can judge for the first time that he/she no longer hears any Korotkoff sound Kc after a delay of one pulse beat equivalent Δt from a time t20 at which he/she actually heard the last Korotkoff sound Kce. Therefore, at the time t2 when the observer pressed the HOLD button, the cuff pressure Pc is already a value lowered by one pulse beat equivalent ΔP from the true minimum blood pressure DIA. It is considered that this is a reason why an obtained minimum blood pressure is evaluated (recorded) particularly lower than an actual blood pressure (auscultatory value) when the hold function of the electronic sphygmomanometer is used. This analysis was carried out independently by the present inventor.

Therefore, an object of this invention is to provide a blood pressure recording device configured to measure and record a blood pressure based on a Korotkoff sound generated by an artery through a measurement target site of a subject, the blood pressure recording device being able to accurately record a diastolic blood pressure value (minimum blood pressure) when an observer operates an operation unit (e.g., such as the above HOLD button) based on the Korotkoff sound. An object of this invention is to provide a blood pressure recording method for measuring and recording blood pressure using such a blood pressure recording device, the blood pressure recording method being able to accurately record a diastolic blood pressure value (minimum blood pressure).

In order to achieve the object, in a first aspect, a blood pressure recording device of the present disclosure is a blood pressure recording device configured to measure and record a blood pressure based on a Korotkoff sound generated by an artery through a measurement target site of a subject, the blood pressure recording device comprising:

a cuff worn around the measurement target site;

a cuff pressure adjustment unit for pressurizing or depressurizing a cuff pressure, the cuff pressure being a pressure of the cuff;

a pressure detection unit that detects the cuff pressure in a depressurization process of the cuff pressure by the cuff pressure adjustment unit;

a time detection unit that detects a time at which a waveform feature point of a pulse beat was generated for each pulse beat of a pulse wave indicated by the artery through the measurement target site in the depressurization process;

a first data accumulation unit that causes a memory to sequentially store, as a data pair, a time at which the waveform feature point of the pulse beat was generated or a sign corresponding to the time and the cuff pressure at the time when the waveform feature point of the pulse beat was generated, in association with each other in the depressurization process;

an indicator;

a first display processing unit that performs processing of causing the indicator to display the cuff pressure detected by the pressure detection unit; and

an operation unit configured to be operated by an observer who listens to a Korotkoff sound generated by the artery through the measurement target site with a stethoscope while viewing the cuff pressure displayed on the indicator by the first display processing unit in the depressurization process, wherein

the operation unit is configured to input a first timing signal indicating a time at which the observer judged that the Korotkoff sound had started generating and/or a second timing signal indicating a time at which the observer judged that the Korotkoff sound had stopped generating, and

the blood pressure recording device includes a recording processing unit that specifies a data pair corresponding to one pulse beat before a data pair corresponding to a time indicated by the second timing signal among a series of data pairs stored in the memory in the depressurization process, and records, as a corrected diastolic blood pressure value, the cuff pressure included in the specified data pair.

In the present description, the “subject” refers to a person whose blood pressure is to be measured, typically a patient. The “observer” refers to a person who intends to measure and record the blood pressure, typically, a medical worker such as a medical doctor or a nurse.

The “Korotkoff sound” refers to a sound generated by an artery through a measurement target site for each pulse beat in a depressurization process of cuff pressure. The “measurement target site” typically refers to an upper arm of the subject.

The “waveform feature point” of a pulse beat of a pulse wave refers to a feature point that can specify a timing in the pulse beat, such as a rising point indicating the rise of the pulse beat or a peak point indicating a peak (top of the mountain) of the pulse beat.

The “sign corresponding to the time” at which the waveform feature point was generated indicates a serial number (pulse beat number) assigned to each pulse beat, for example.

The “operation unit” widely includes means to allow an observer to input a timing signal, such as a switch that is temporarily turned on at a timing when pressed such as a HOLD button in known examples, or a touch key that is temporarily turned on at a timing when touched.

The “data pair corresponding to a time indicated by . . . timing signal” refers to a data pair stored immediately before (specifically, immediately before by a reaction time of the observer) the time indicated by the timing signal among a series of data pairs stored in the memory in the depressurization process.

In a second aspect, a blood pressure recording method of the present disclosure is a blood pressure recording method for measuring and recording a blood pressure of a measurement target site of a subject using the blood pressure recording device according to claim 1, the blood pressure recording method comprising:

wearing the cuff around the measurement target site, and bringing the stethoscope into contact with a part through which the artery of the measurement target site passes;

after pressurizing the cuff pressure by the cuff pressure adjustment unit to temporarily stop a blood flow in the artery through the measurement target site, in the depressurization process of the cuff pressure,

detecting the cuff pressure by the pressure detection unit;

detecting, by time detection unit, a signal representing the time at which the waveform feature point of the pulse beat was generated for each pulse beat of the pulse wave indicated by the artery through the measurement target site;

causing, by the first data accumulation unit, the memory to sequentially store, as the data pair, the time at which the waveform feature point of the pulse beat was generated or the sign corresponding to the time and the cuff pressure at the time when the waveform feature point of the pulse beat was generated, in association with each other;

inputting the second timing signal indicating at least the time at which the observer judged that the Korotkoff sound had stopped generating, by operating the operation unit by the observer who listens to the Korotkoff sound generated by the artery through the measurement target site with the stethoscope while viewing the cuff pressure displayed on the indicator by the first display processing; and

during the depressurization process or after the depressurization process ending, by the recording processing unit, specifying the data pair corresponding to one pulse beat before the data pair corresponding to the time indicated by the second timing signal among the series of data pairs stored in the memory in the depressurization process, and recording, as the corrected diastolic blood pressure value, the cuff pressure included in the specified data pair.

In a third aspect, a blood pressure recording device of the present disclosure is a blood pressure recording device configured to measure and record a blood pressure based on a Korotkoff sound generated by an artery through a measurement target site of a subject, the blood pressure recording device comprising:

a cuff worn around the measurement target site;

a cuff pressure adjustment unit for pressurizing or depressurizing a cuff pressure, the cuff pressure being a pressure of the cuff;

a pressure detection unit that detects the cuff pressure in a depressurization process of the cuff pressure by the cuff pressure adjustment unit;

a microphone that converts a sound generated by the artery through the measurement target site into a sound signal that is an electric signal and outputs the sound signal in the depressurization process;

a Korotkoff sound signal detection unit that detects a Korotkoff sound signal indicating a Korotkoff sound from the sound signal output by the microphone;

a second data accumulation unit that causes a memory to sequentially store, as a data pair, a time at which the Korotkoff sound signal was detected or a sign corresponding to the time and the cuff pressure at the time when the Korotkoff sound signal was detected, in association with each other in the depressurization process;

an indicator;

a first display processing unit that performs processing of causing the indicator to display the cuff pressure detected by the pressure detection unit; and

an operation unit configured to be operated by an observer who listens to the Korotkoff sound generated by the artery through the measurement target site with a stethoscope or listens to the Korotkoff sound indicated by the Korotkoff sound signal with an electro-acoustic conversion device while viewing the cuff pressure displayed on the indicator by the first display processing unit in the depressurization process, wherein

the operation unit is configured to input a first timing signal indicating a time at which the observer judged that the Korotkoff sound had started generating and/or a second timing signal indicating a time at which the observer judged that the Korotkoff sound had stopped generating, and

the blood pressure recording device includes a recording processing unit that specifies a data pair stored last before the time indicated by the second timing signal among a series of data pairs stored in the memory in the depressurization process, and records, as a corrected diastolic blood pressure value, the cuff pressure included in the specified data pair.

In the present description, the “Korotkoff sound signal” refers to a pulse-like electric signal indicating a Korotkoff sound. The “electro-acoustic conversion device” refers to a device that converts an electric signal into sound, for example, a speaker, a headphone, an earphone, or the like.

In a fourth aspect, a blood pressure recording method of the present disclosure is a blood pressure recording method for measuring and recording a blood pressure of a measurement target site of a subject using the blood pressure recording device according to claim 10, the blood pressure recording method comprising:

wearing the cuff around the measurement target site;

arranging the microphone so as to acquire the sound generated by the artery through the measurement target site, or in addition to arrangement of the microphone, bringing a stethoscope into contact with a part through which the artery of the measurement target site passes;

after pressurizing the cuff pressure by the cuff pressure adjustment unit to temporarily stop a blood flow in the artery through the measurement target site, in the depressurization process of the cuff pressure,

detecting the cuff pressure by the pressure detection unit;

converting, by the microphone, the sound generated by the artery through the measurement target site into the sound signal and outputting the sound signal;

detecting, by the Korotkoff sound signal detection unit, the Korotkoff sound signal from the sound signal output by the microphone;

causing, by the second data accumulation unit, the memory to sequentially store, as the data pair, the time at which the Korotkoff sound signal was detected or the sign corresponding to the time and the cuff pressure at the time when the Korotkoff sound signal was detected, in association with each other;

inputting the second timing signal indicating at least the time at which the observer judged that the Korotkoff sound had stopped generating, by operating the operation unit by the observer who listens to the Korotkoff sound generated by the artery through the measurement target site with the stethoscope or listens to the Korotkoff sound indicated by the Korotkoff sound signal with the electro-acoustic conversion device while viewing the cuff pressure displayed on the indicator by the first display processing; and

during the depressurization process or after the depressurization process ending, by the recording processing unit, specifying the data pair stored last before the time indicated by the second timing signal among the series of data pairs stored in the memory in the depressurization process, and recording, as the corrected diastolic blood pressure value, the cuff pressure included in the specified data pair.

### BRIEF DESCRIPTION OF THE DRAWINGS

FIG. 1 is a view showing a block configuration of a sphygmomanometer as a first embodiment of a blood pressure recording device of this invention.

FIG. 2 is a view showing an example of a situation when blood pressure is measured using the sphygmomanometer.

FIG. 3 is a view showing a flow of a blood pressure recording method of an embodiment using the sphygmomanometer of FIG. 1.

FIG. 4 is a view illustrating how to measure and record a systolic blood pressure value and a diastolic blood pressure value by the flow of the blood pressure recording method of FIG. 3.

FIG. 5A is a scatter diagram where the auscultatory value is represented on the horizontal axis and the difference (HOLD value−auscultatory value) is represented on the vertical axis, for the systolic blood pressure value (maximum blood pressure) recorded by the flow of the blood pressure recording method of FIG. 3. FIG. 5B is a scatter diagram where the auscultatory value is represented on the horizontal axis and the difference (HOLD value−auscultatory value) is represented on the vertical axis, for the diastolic blood pressure value (minimum blood pressure) recorded by the flow of the blood pressure recording method of FIG. 3.

FIG. 6 is a view showing a block configuration of a sphygmomanometer as a second embodiment of the blood pressure recording device of this invention.

FIG. 7 is a view showing a flow of a blood pressure recording method of an embodiment using the sphygmomanometer of FIG. 6.

FIG. 8 is a view showing a block configuration of a sphygmomanometer as a third embodiment of the blood pressure recording device of this invention.

FIG. 9 is a view showing a flow of a blood pressure recording method of an embodiment using the sphygmomanometer of FIG. 8.

FIG. 10 is a view illustrating how to select the cuff pressure displayed as an option on the indicator after the depressurization process of the cuff pressure ending.

FIG. 11 is a view showing an example of a data set stored in the memory by the flow of the blood pressure recording method of FIG. 9.

FIG. 12 is a view showing a flow of a modification in which the flow of the blood pressure recording method of FIG. 9 is modified.

FIG. 13 is a view showing a block configuration of a sphygmomanometer as a fourth embodiment of the blood pressure recording device of this invention.

FIG. 14 is a view showing a flow of a blood pressure recording method of an embodiment using the sphygmomanometer of FIG. 13.

FIG. 15 is a view illustrating how to measure and record a systolic blood pressure value and a diastolic blood pressure value by the flow of the blood pressure recording method of FIG. 14.

FIG. 16 is a view showing a block configuration of a sphygmomanometer of a modification in which the sphygmomanometer of FIG. 13 is modified.

FIG. 17 is a view showing a block configuration of a blood pressure measurement system as a fifth embodiment of the blood pressure recording device of this invention.

FIG. 18 is a view showing a part (first half) of the flow of the blood pressure recording method of an embodiment by the blood pressure measurement system of FIG. 17.

FIG. 19 is a view showing a remaining part (second half) of the flow of the blood pressure recording method of an embodiment by the blood pressure measurement system of FIG. 17.

FIGS. 20A, 20B, and 20C are views each showing a modification that an indicator constituting a sphygmomanometer (and blood pressure measurement system) can have.

FIG. 21 is a view illustrating how to record a maximum blood pressure and a minimum blood pressure using a hold function of the electronic sphygmomanometer of a conventional example.

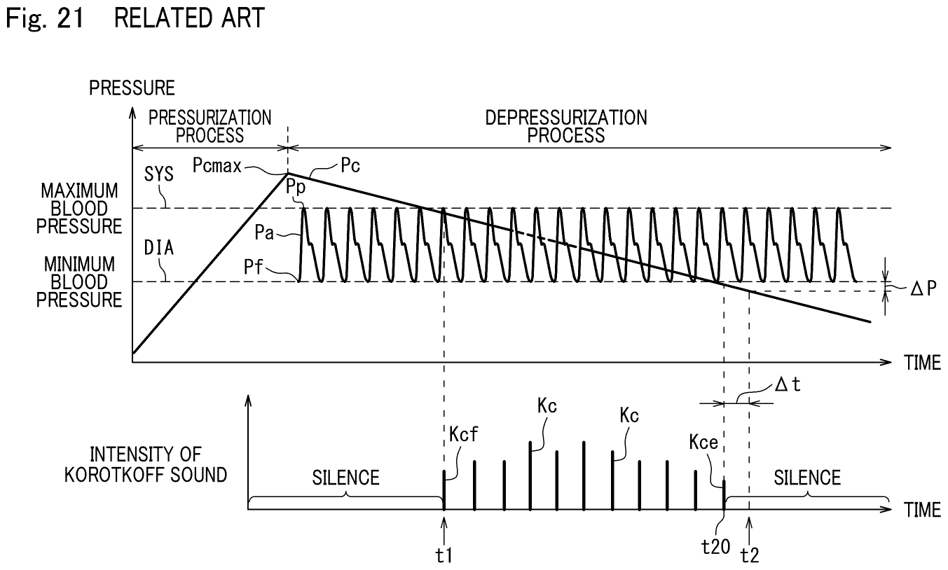

FIG. 22A is a scatter diagram where the auscultatory value is represented on the horizontal axis and the difference (HOLD value−auscultatory value) is represented on the vertical axis, for the maximum blood pressure recorded by the hold function of the electronic sphygmomanometer of the conventional example. FIG. 22B is a scatter diagram where the auscultatory value is represented on the horizontal axis and the difference (HOLD value−auscultatory value) is represented on the vertical axis, for the minimum blood pressure recorded by the hold function of the electronic sphygmomanometer of the conventional example.

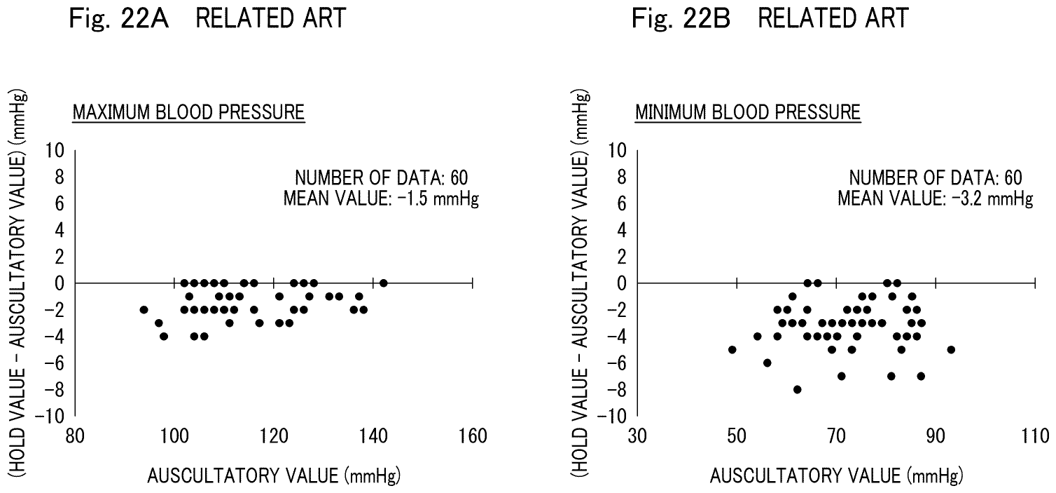

### DETAILED DESCRIPTION

Embodiments of this invention will be described below in detail with reference to the drawings.

### First Embodiment

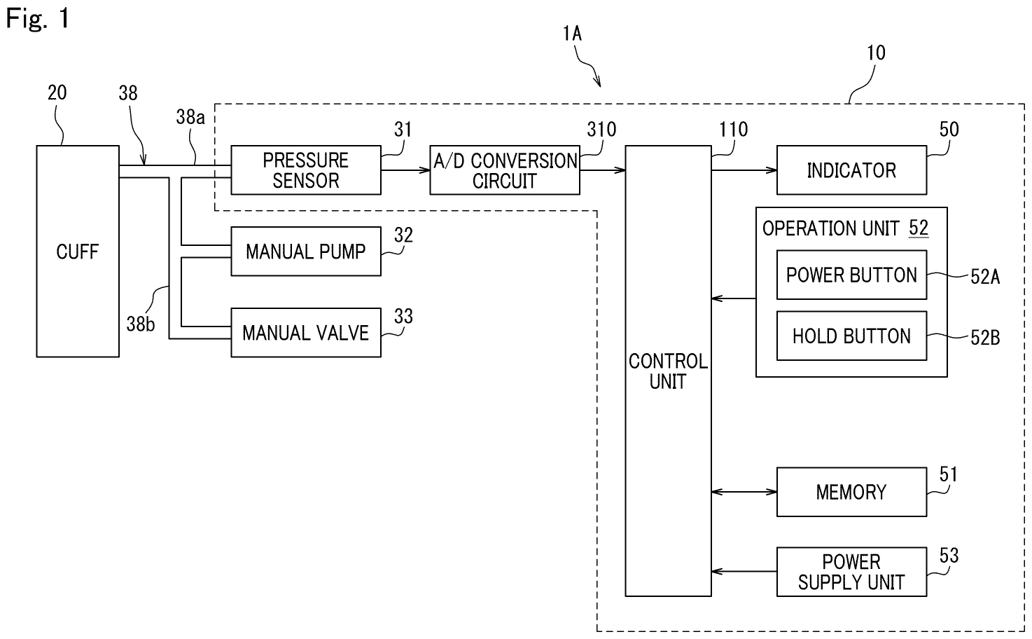

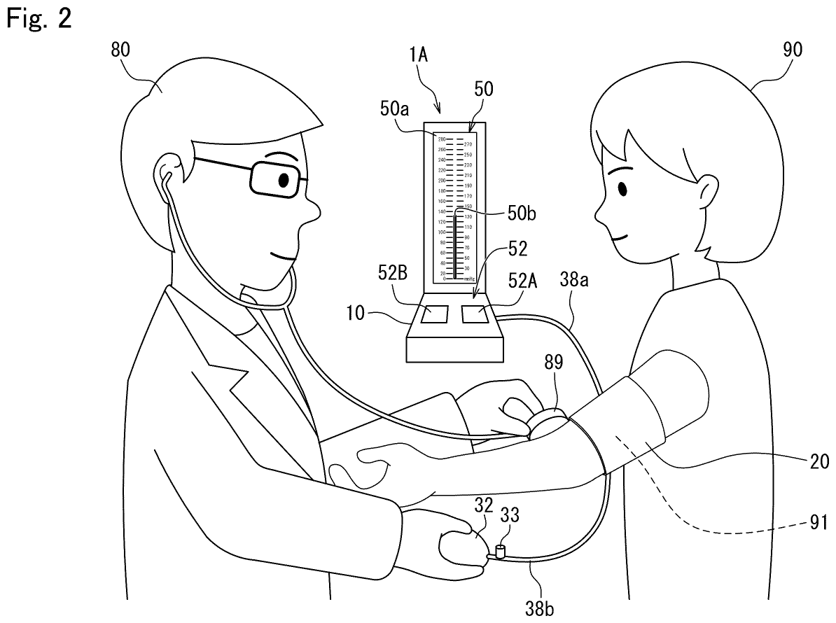

FIG. 1 is a view showing a block configuration of a sphygmomanometer 1A as the first embodiment of the blood pressure recording device of this invention. FIG. 2 shows a situation when an observer (in this example, a medical doctor) 80 measures a blood pressure of an upper arm 91 as a measurement target site of a subject (in this example, a patient) 90 using the sphygmomanometer 1A. The sphygmomanometer 1A is configured to measure and record blood pressure based on a Korotkoff sound Kc generated by an artery through the upper arm 91 of the subject 90.

As shown in FIG. 2, the sphygmomanometer 1A roughly includes a main body 10 including an indicator 50, a cuff 20 worn around the upper arm 91 of the subject 90, and a manual pump (air bulb) 32 and a manual valve 33 as a pressure adjustment unit.

The cuff 20 is a general cuff used for blood pressure measurement of the upper arm 91, and includes an air bag (not shown) for pressurizing the upper arm 91. The main body 10 and the cuff 20 are connected by an air pipe 38a having flexibility. The cuff 20, the manual pump 32, and the manual valve 33 are connected by an air pipe 38b having flexibility. The manual pump 32 and the manual valve 33 are also general ones used for a blood pressure measurement.

As shown in FIG. 1, the main body 10 includes a control unit 110, the indicator 50, an operation unit 52, a memory 51, a power supply unit 53, a pressure sensor 31 as a pressure detection unit, and an A/D conversion circuit 310. In this example, the air pipe 38a is connected to the pressure sensor 31 in a fluid flowable manner. In this example, the air pipe 38a merges with the air pipe 38b connected to the manual pump 32 and the manual valve 33, and is connected to the cuff 20 in a fluid flowable manner as one air pipe 38.

In this example, as shown in FIG. 2, the indicator 50 includes a liquid crystal display (LCD) erected in a rear part of the main body 10, and displays predetermined information in accordance with a control signal from the control unit 110. In this example, the indicator 50 is configured to display, as an image, a scale 50a arranged in the vertical direction and a bar graph 50b extending and contracting along the scale 50a in combination in a form imitating a traditional mercury sphygmomanometer, and display the pressure (this is called “cuff pressure Pc”) of the cuff 20 during blood pressure measurement. Together with this, the indicator 50 may be configured to digitally display the cuff pressure Pc being measured and/or an obtained blood pressure value. Note that the indicator 50 may include an organic electro luminescence (EL) display or may include a light emitting diode (LED).

As shown in FIG. 1, the operation unit 52 includes a power button 52A and a HOLD button 52B in this example, and is configured to input, to the control unit 110, a signal corresponding to an operation of an observer 80, who is a user. For convenience of operation, as shown in FIG. 2, the power button 52A and the HOLD button 52B are provided on the upper surface of the main body 10. Specifically, the power button 52 is a latch type (self-holding type) switch in this example, and maintains an ON state when pressed once, and turns off when pressed once again. Due to this, when the power button 52A is pressed once, the power of the sphygmomanometer 1A is turned on, and when the power button 52A is pressed once again, the power of the sphygmomanometer 1A is turned off. On the other hand, the HOLD button 52B is a momentary type (self-return type) switch in this example, and is brought into the on state only while being pressed down, and returns to the off state when being released. As described in detail later, when pressed down by the observer 80 who listens to the Korotkoff sound Kc generated from the artery through the upper arm 91 with a stethoscope 89 while viewing the cuff pressure Pc displayed in real time as the bar graph 50b on the indicator 50, the HOLD button 52B is used to input a timing signal (this is called a “first timing signal”) indicating a time at which the observer 80 judged that the Korotkoff sound Kc had started generating, and/or to input a timing signal (this is called a “second timing signal”) indicating a time at which the observer 80 judged that the Korotkoff sound Kc had stopped generating.

The memory 51 shown in FIG. 1 stores data of a program for controlling the sphygmomanometer 1A, setting data for setting various functions of the sphygmomanometer 1A, data during blood pressure measurement, data of a blood pressure measurement result, and the like. The memory 51 is also used as a work memory or the like when a program is executed.

The control unit 110 includes a central processing unit (CPU) as a processor, and controls the entire operation of the sphygmomanometer 1A. Specifically, the control unit 110 performs control for measuring and recording the blood pressure in accordance with a program for controlling the sphygmomanometer 1A stored in the memory 51. A specific method of measuring and recording the blood pressure will be described later.

The pressure sensor 31 is a piezoresistive pressure sensor in this example, and converts, into a voltage, a change in electric resistance due to a piezoresistive effect generated by the pressure (cuff pressure Pc) of the cuff 20 received through the air pipes 38 and 38a. The A/D conversion circuit 310 converts and transmits, to the control unit 110, the voltage from the pressure sensor 31 from an analog value to a digital value. The control unit 110 obtains the cuff pressure Pc in accordance with the voltage.

As described above, the manual pump 32 and the manual valve 33 are general ones used for a manual blood pressure measurement. When the manual pump 32 is operated in a state where the manual valve 33 is closed, air is supplied from the manual pump 32 to the cuff 20 via the air pipes 38b and 38, and the cuff pressure Pc is pressurized. When the manual valve 33 is opened, the air is exhausted from the cuff 20 to the atmosphere through the air pipes 38 and 38b and the manual valve 33, and the cuff pressure Pc is depressurized.

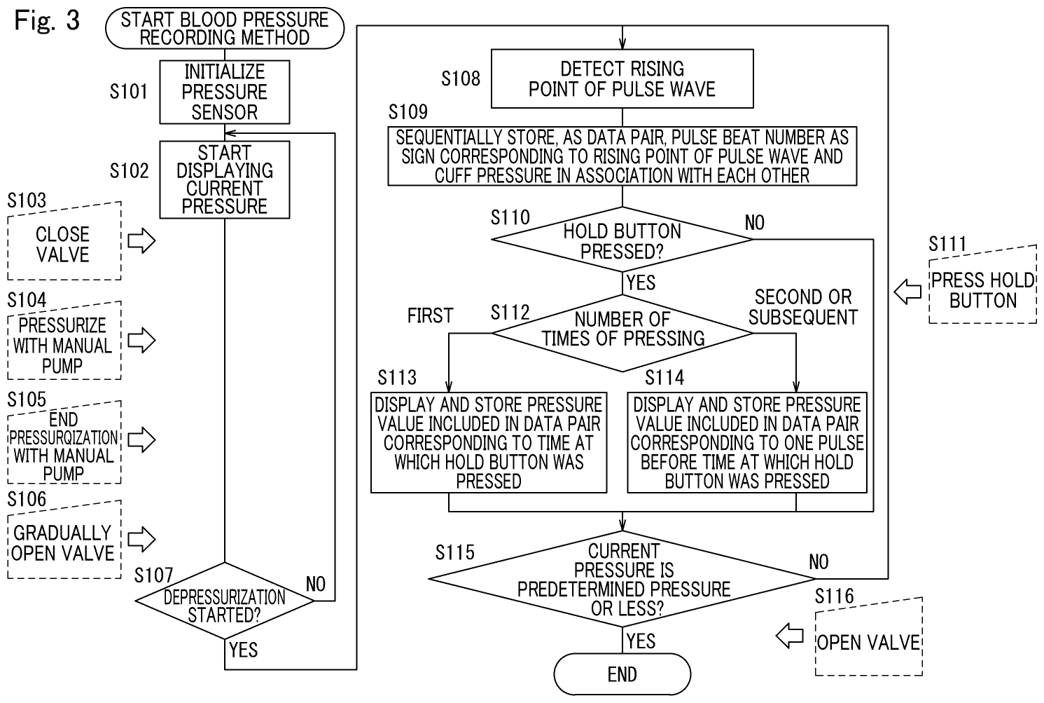

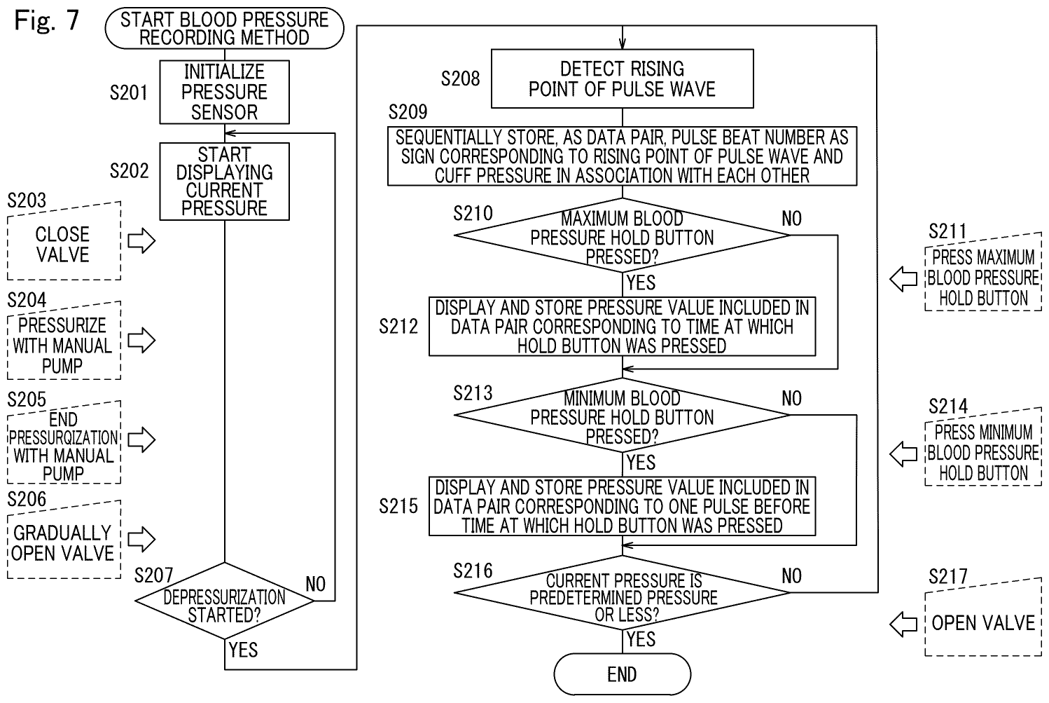

The power supply unit 53 supplies or cuts off power to each unit in the main body 10 such as the control unit 110, the indicator 50, the memory 51, the pressure sensor 31, and other units in response to ON/OFF of the power button 52A. FIG. 3 shows a flow of a blood pressure recording method of an embodiment in which the observer 80 measures and records the blood pressure of the upper arm 91 of the subject 90 using the sphygmomanometer 1A. Note that in FIG. 3 (and FIGS. 7, 9, 12, 14, and 19 described later), for easy understanding, the processing and determination performed by the control unit 110 in the main body 10 are drawn with a solid line outline, and manual operation and manual input by the observer 80 are drawn with a broken line outline in a form of being arranged next to a flow of the solid line.

As preparation for blood pressure measurement, this example assumes that the observer 80 puts the cuff 20 on around the upper arm 91 of the subject 90, and brings the stethoscope 89 into contact with a part of the upper arm 91 of the subject 90 where the artery passes (a part corresponding to downstream of the cuff 20). The manual valve 33 is assumed to be opened.

In this state, when the observer 80 turns on the power button 52A of the sphygmomanometer 1A, the control unit 110 in the main body 10 initializes a processing memory area and initializes the pressure sensor 31 (step S101 of FIG. 3). Specifically, 0 mmHg adjustment (the atmospheric pressure is set to 0 mmHg) of the pressure sensor 31 is performed in a state where the manual valve 33 is opened.

Subsequently, in step S102, the control unit 110 acts as a first display processing unit, and starts processing of causing the indicator 50 to display in real time, as the bar graph 50b along the scale 50a, a current cuff pressure Pc detected by the pressure sensor 31. Initially, 0 mmHg is displayed.

In this state, the observer 80 closes the manual valve 33 (step S103), and temporarily pressurizes the cuff pressure Pc to Pcmax higher than an expected maximum blood pressure by about 30 mmHg by the manual pump 32 (steps S104 and S105). Due to this, a blood flow through the artery of the upper arm 91 is temporarily stopped. Subsequently, in step S106, the observer 80 gradually opens the manual valve 33 and starts a depressurization of the cuff pressure Pc at a depressurization rate of typically 2 to 3 mmHg/sec. As shown in step S107, until the depressurization is started, the control unit 110 waits for the depressurization to start while causing the indicator 50 to display the current cuff pressure Pc. Here, whether or not the depressurization has been started can be determined, for example, by the control unit 110 calculating a first derivative (dPc/dt) with time t of the cuff pressure Pc detected by the pressure sensor 31, and whether or not the first derivative (dPc/dt) becomes negative.

When the depressurization is started (Yes in step S107), the control unit 110 performs the processing of steps S108 to S114 in this depressurization process.

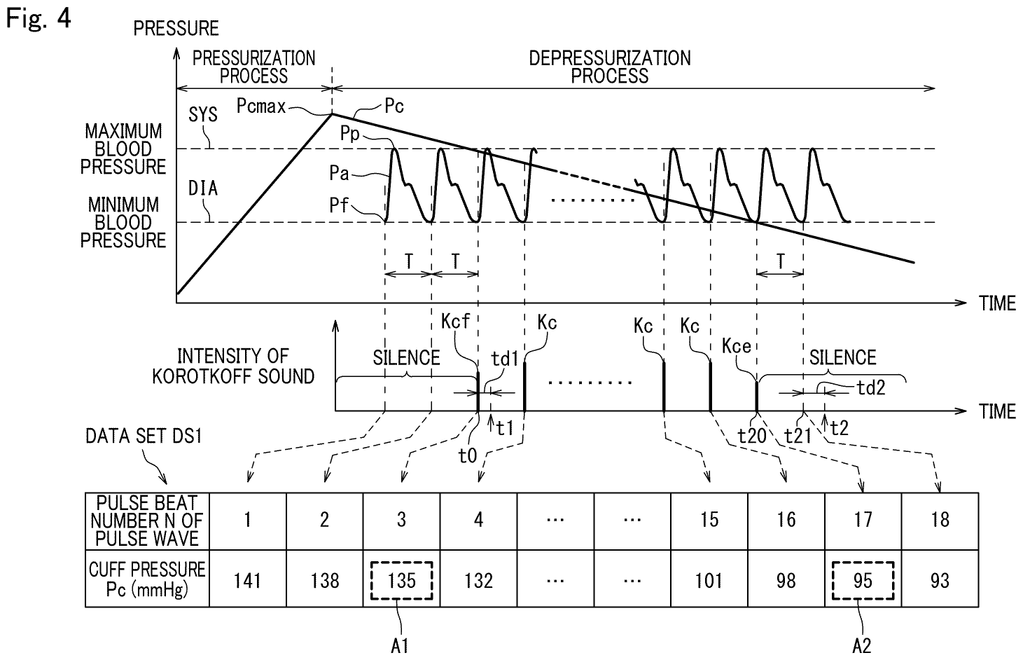

Here, the upper part of FIG. 4 shows a transition of the intra-arterial pressure Pa and a change in the cuff pressure Pc with the lapse of time. In this example, the intra-arterial pressure Pa shows, between the minimum blood pressure DIA and the maximum blood pressure SYS, a mountain-like waveform that rises with a rising point Pf, shows a peak with a peak point Pp, and falls, for each pulse beat of the pulse wave (in this example, the period is T). As described above, the cuff pressure Pc is temporarily pressurized to Pcmax higher than an expected maximum blood pressure by about 30 mmHg in the pressurization process, and then, in the depressurization process, the cuff pressure Pc is depressurized at a depressurization rate of 2 to 3 mmHg/sec in this example. Then, when the cuff pressure Pc falls below the maximum blood pressure SYS, as shown in the middle part of FIG. 4, a Korotkoff sound Kc starts generating for each pulse beat, and when the cuff pressure Pc further falls below the minimum blood pressure DIA, the Korotkoff sound Kc stops generating. Note that in this depressurization process, Kcf indicates a first Korotkoff sound, and Kce indicates a last Korotkoff sound.

In this depressurization process, the control unit 110 acts as a time detection unit, and first, in step S108 of FIG. 3, starts to obtain a time at which the rising point Pf (see the upper part of FIG. 4) as the waveform feature point of the pulse beat was generated, for each pulse beat of the pulse wave. The rising point Pf of the pulse beat can be obtained, for example, by the control unit 110 extracting a fluctuation component (Pm) of the cuff pressure Pc detected by the pressure sensor 31, calculating a first derivative (dPm/dt) and a second derivative (d²Pm/dt²) of the fluctuation component Pm with time t, and detecting a point at which the sign of the first derivative (dPm/dt) changes from negative to positive and the second derivative (d²Pm/dt²) becomes zero.

Note that as the “waveform feature point” of the pulse beat of the pulse wave, the peak point Pp indicating a peak (top of a mountain) of the pulse beat may be used in place of the rising point Pf indicating the rise of the pulse beat.

Concurrently with this processing, in step S109 of FIG. 3, the control unit 110 acts as a first data accumulation unit, and starts processing of causing the memory 51 to sequentially store, as a data pair, a pulse beat number N as a sign corresponding to the time at which the rising point Pf of the pulse beat was generated and the cuff pressure Pc at the time when the rising point Pf of the pulse beat was generated, in association with each other. In this example, as shown in the lower part of FIG. 4, as a series of data pairs (this is called a “data set”) DS1 obtained in this depressurization process, a data pair with the pulse beat number N=1 and the cuff pressure Pc=141 (mmHg) is stored, then a data pair with the pulse beat number N=2 and the cuff pressure Pc=138 (mmHg) is stored, and in this manner, a data pair with the pulse beat number N=18 and the cuff pressure Pc=93 (mmHg) is finally stored. Since this processing is performed without depending on the operation of the observer 80, this data set DS1 is stored without being affected by a reaction time (described later) when the observer 80 presses the HOLD button 52B.

Note that the time at which the rising point Pf of the pulse beat was generated and the cuff pressure Pc at the time when the rising point Pf of the pulse beat was generated may be directly associated with each other and sequentially stored in the memory 51 as a data pair. However, use of the pulse beat number N as described above enables resources of the memory 51 to be saved.

As the waveform feature point of the pulse beat, the peak point Pp may be used in place of the rising point Pf of the pulse beat. The peak point Pp of the pulse beat can be obtained, for example, by the control unit 110 extracting a fluctuation component (Pm) through a filter from the cuff pressure Pc detected by the pressure sensor 31, calculating a first derivative (dPm/dt) and a second derivative (d²Pm/dt²) of the fluctuation component Pm with time t, and detecting a point at which the sign of the first derivative (dPm/dt) changes from positive to negative and the second derivative (d²Pm/dt²) becomes zero.

While the processing in steps S108 and S109 of FIG. 3 is being performed in the depressurization process, the control unit 110 determines in step S110 whether or not the HOLD button 52B is pressed. When the HOLD button 52B is not pressed (No in step S110), the control unit 110 continues the processing of steps S108 to S110 as long as the current cuff pressure Pc is higher than a predetermined pressure (in this example, 30 mmHg) (No in step S115). Note that the above 30 mmHg is a pressure lower than an assumed diastolic blood pressure value (minimum blood pressure), and is determined in advance as a pressure at which the depressurization process is to be ended.

During the depressurization process, the observer 80 listens to the Korotkoff sound Kc generated by the artery through the upper arm 91 with the stethoscope 89 while viewing the cuff pressure Pc displayed in real time as the bar graph 50b on the indicator 50 shown in FIG. 2. Then, in step S111 of FIG. 3, the observer 80 presses the HOLD button 52 at a time t1 delayed by a reaction time td1 from a time t0 at which he could judge that the Korotkoff sound Kc had started generating (i.e., a time at which he heard the first Korotkoff sound Kcf from a silent state) as exemplified in the middle part of FIG. 4. This corresponds to the first pressing in the depressurization process. Here, the observer 80 can judge that the Korotkoff sound Kc has started generating (and judge that the Korotkoff sound Kc has stopped generating), with the same sense as viewing a traditional mercury sphygmomanometer. Therefore, it can be said that it is convenient for an observer who is accustomed to using a traditional mercury sphygmomanometer. Note that it is assumed that the reaction time td1 of the observer 80 normally does not exceed 1 pulse beat T.

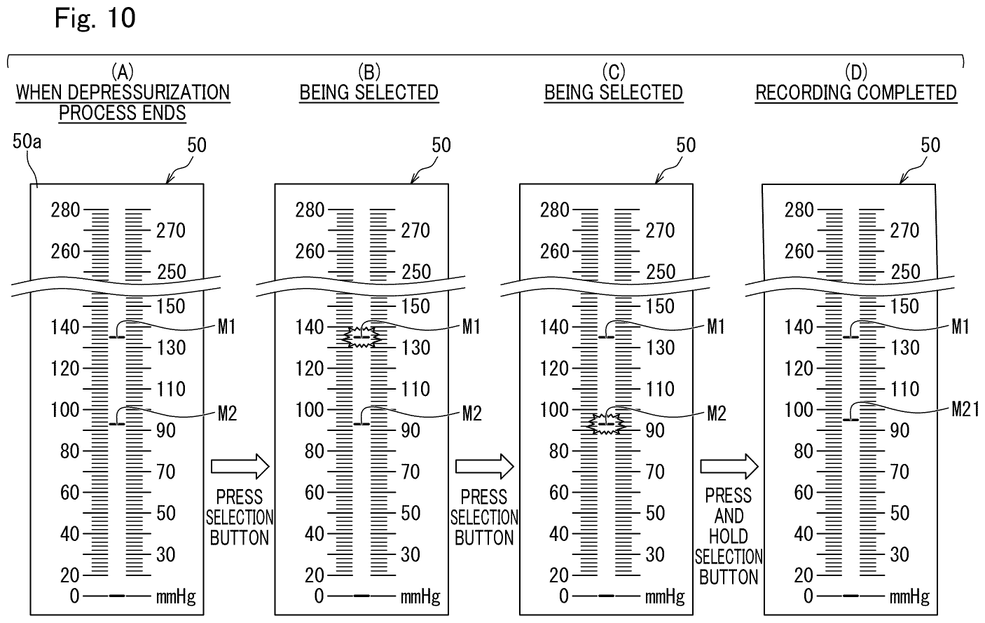

When the HOLD button 52B is pressed (Yes in step S110 of FIG. 3), the control unit 110 acts as a recording processing unit, and determines whether a number of times of pressing of the HOLD button 52B in the depressurization process is a first time or a second or subsequent time in step S112 of FIG. 3. In this example, the memory 51 includes a counter (not shown) that counts the number of times of pressing of the HOLD button 52B. This counter is reset every time the depressurization process starts, and counts the number of times of pressing of the HOLD button 52B. By referring to the count value of this counter, the control unit 110 can determine whether the number of times of pressing of the HOLD button 52B is the first time or the second or subsequent time. Here, if the number of times of pressing of the HOLD button 52B is the first time, in step S113, the control unit 110 determines that a first timing signal t1 (for simplicity, represented by the same reference sign as the time t1) was input at the time t1 at which the first pressing was performed as exemplified in the middle part of FIG. 4. Then, the control unit 110 acts as the recording processing unit, and specifies a data pair (in the example in the lower part of FIG. 4, the data pair with the pulse beat number N=3 stored immediately before the time t1) corresponding to the time indicated by the first timing signal t1 among the data set DS1 stored in the memory 51. Furthermore, the control unit 110 causes the memory 51 to store, as a systolic blood pressure value, the cuff pressure Pc included in the specified data pair. In the example in the lower part of FIG. 4, 135 mmHg (indicated by a broken line frame A1) included in the data pair with the pulse beat number N=3 is stored as the systolic blood pressure value. Together with this, in this example, the obtained systolic blood pressure value 135 mmHg is displayed on the indicator 50. This display may be displayed as a digital value on the indicator 50 as, for example, “Maximum Blood Pressure 135 mmHg”, and/or may be displayed as a horizontal bar-shaped mark indicating a pressure value by a position along the scale 50a, such as a mark M1 shown in FIG. 10 described later. This enables the observer 80 to easily recognize the obtained systolic blood pressure value by viewing the indicator 50. Note that at this time point, the data set DS1 only stores the data pair with the pulse beat number N=1 to the data pair with the pulse beat number N=3.

Thereafter, the depressurization process proceeds, and the cuff pressure Pc decreases. In step S111 of FIG. 3 again, the observer 80 presses the HOLD button 52 at a time t2 delayed by a reaction time td2 from a time t21 at which he could judge that the Korotkoff sound Kc had stopped generating (i.e., a time at which he could judge for the first time that he no longer heard any Korotkoff sound after being delayed by 1 pulse beat T from a time t20 at which he could hear the last Korotkoff sound Kce) as exemplified in the middle part of FIG. 4. This corresponds to the second pressing in the depressurization process. Note that it is assumed that the reaction time td2 of the observer 80 normally does not exceed 1 pulse beat T.

When the HOLD button 52B is pressed (Yes in step S110 of FIG. 3), the control unit 110 determines whether the number of times of pressing of the HOLD button 52B in the depressurization process is the first time or the second or subsequent time, by referring to the count value of the counter in step S112 of FIG. 3. Here, if the number of times of pressing of the HOLD button 52B is the second time, in step S114, the control unit 110 determines that a second timing signal t2 (for simplicity, represented by the same reference sign as the time t2) was input at the time t2 at which the second pressing was performed as exemplified in the middle part of FIG. 4. Then, the control unit 110 acts as the recording processing unit, and specifies a data pair (in the example in the lower part of FIG. 4, a data pair with the pulse beat number N=18 stored immediately before time t2) corresponding to the time indicated by the second timing signal t2 among the data set DS1 stored in the memory 51, and further specifies a data pair (in the example in the lower part of FIG. 4, the data pair with the pulse beat number N=17) corresponding to one pulse beat before the data pair. Furthermore, the control unit 110 causes the memory 51 to store, as a corrected diastolic blood pressure value, the cuff pressure Pc included in the latter data pair. In the example in the lower part of FIG. 4, 95 mmHg (indicated by a broken line frame A2) included in the data pair with the pulse beat number N=17 is stored as the corrected diastolic blood pressure value. Together with this, in this example, the control unit 110 acts as a third display processing unit, and causes the indicator 50 to display the corrected diastolic blood pressure value 95 mmHg. This display may be displayed as a digital value on the indicator 50 as, for example, “Minimum Blood Pressure 95 mmHg”, and/or may be displayed as a horizontal bar-shaped mark indicating a pressure value by a position along the scale 50a, such as a mark M2 shown in FIG. 10 described later. This enables the observer 80 to easily recognize the corrected diastolic blood pressure value by viewing the indicator 50.

Note that the second “or subsequent” in step S112 of FIG. 3 is configured to be able to cope with the possibility that the HOLD button 52B is pressed three times or more during the depressurization process. Specifically, if the subject 90 exhibits arrhythmias, the pulse beat may be interrupted midway. In a case of a pregnant woman, the Korotkoff sound may continue to be heard down to a low pressure (e.g., 30 mmHg). The sound quality of the Korotkoff sound varies in five stages from generation to stop. Each stage of sound is called a “phase”, and there are phase I to phase V. The point (beginning of phase IV) at which the sound quality of the Korotkoff sound changes and the last sound of the Korotkoff sound may be both recorded. Therefore, as described above, it is configured to be able to cope with the possibility that the HOLD button 52B is pressed three times or more during the depressurization process.

Thereafter, when the depressurization process proceeds and the current cuff pressure Pc becomes a predetermined pressure (in this example, 30 mmHg) or less (Yes in step S115), the control unit 110 ends the processing of the flow of this blood pressure recording method. The observer 80 opens the manual valve 33 to decrease the cuff pressure Pc (step S116). Note that the observer 80 may open the manual valve 33 to decrease the cuff pressure Pc as soon as the corrected diastolic blood pressure value is recorded.

In this manner, in this sphygmomanometer 1A, the control unit 110 acts as the recording processing unit, specifies the data pair corresponding to the time indicated by the second timing signal t2 among the series of data pairs stored in the memory 51 in the depressurization process, and records, as the corrected diastolic blood pressure value, the cuff pressure Pc included in the data pair corresponding to one pulse beat before the specified data pair. According to the analysis of the present inventor described above, the corrected diastolic blood pressure value corresponds to not the time t21 at which the observer 80 can judge for the first time that he no longer hears the Korotkoff sound Kc from a state where he continued to hear the Korotkoff sound Kc for each pulse beat but the cuff pressure Pc corresponding to the time t20 at which he actually heard the last Korotkoff sound Kc. Therefore, according to the flow of the blood pressure recording method in FIG. 3 using the sphygmomanometer 1A, it is possible to correct a phenomenon that the minimum blood pressure is evaluated lower by about one pulse beat equivalent.

It is considered that some reaction times td1 and td2, respectively, are required from when the observer judged that the Korotkoff sound Kc had started generating to when he presses the HOLD button 52B, and from when the observer 80 judged that the Korotkoff sound had stopped generating to when he presses the HOLD button 52B. However, in this sphygmomanometer 1A, the control unit 110 acts as the first data accumulation unit in the depressurization process, whereby data pairs are sequentially stored in the memory 51 without being affected by the reaction times td1 and td2. Therefore, the data pair specified by the control unit 110 acting as the recording processing unit is stored without being affected by the reaction times td1 and td2 of the observer 80. It is assumed that the reaction times td1 and td2 of the observer normally do not exceed one pulse beat. As a result, the systolic blood pressure value and the corrected diastolic blood pressure value recorded above are not actually affected by the reaction times td1 and td2 of the observer 80.

Note that for each of the pulse beats, there may be a slight deviation (time interval) between the time at which the rising point Pf of the pulse beat is generated and the time at which the Korotkoff sound Kc of the pulse beat is generated. However, normally, the deviation does not reach half a pulse beat. Therefore, the deviation does not substantially reduce the accuracy of blood pressure measurement (recording).

As described above, according to this first embodiment, the systolic blood pressure value and the diastolic blood pressure value (corrected diastolic blood pressure value) can be accurately recorded as compared with the conventional examples. Moreover, according to this first embodiment, since the control unit 110 determines whether the operation of the operation unit 52 is the first operation or the second operation in the depressurization process, it is possible to quickly record both the systolic blood pressure value and the corrected diastolic blood pressure value. The observer 80 can end the blood pressure measurement as soon as the corrected diastolic blood pressure value can be recorded even in the middle of the depressurization process.

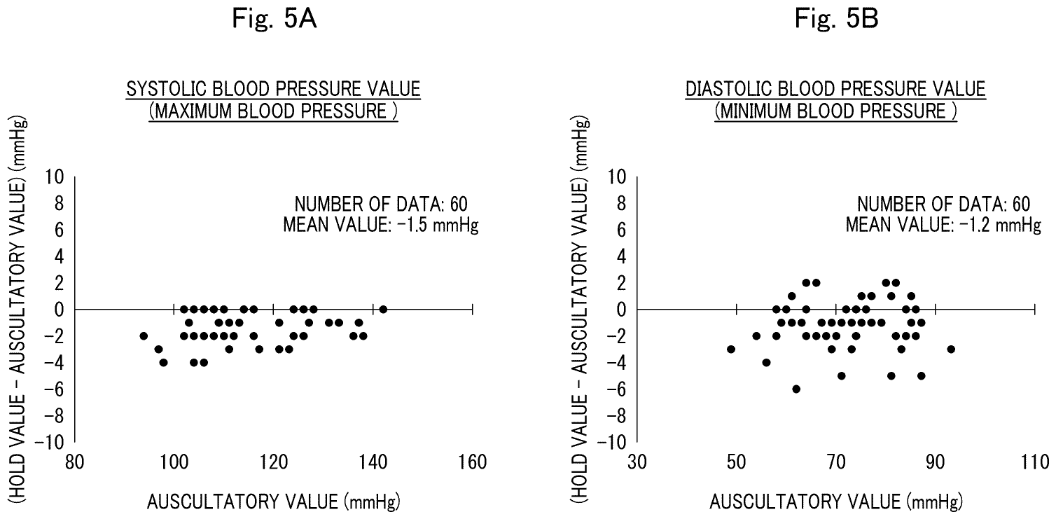

The present inventor validated, by an experiment, the accuracy of the systolic blood pressure value and the corrected diastolic blood pressure value recorded according to this first embodiment. Specifically, using the sphygmomanometer 1A, the present inventor simultaneously performed blood pressure measurement by a normal auscultatory method and blood pressure measurement using the hold function (HOLD button 52B) according to the flow of the blood pressure recording method in FIG. 3. In the blood pressure measurement by the normal auscultatory method, the observer 80 read the maximum blood pressure and the minimum blood pressure based on the Korotkoff sound Kc heard with the stethoscope 89 while viewing the cuff pressure Pc displayed in real time as the bar graph 50b on the indicator 50 (this read value is called the “auscultatory value”). On the other hand, in the blood pressure measurement using the hold function, the observer 80 pressed the HOLD button 52B at the time corresponding to the times t1 and t2 in the middle part of FIG. 4 based on the Korotkoff sound Kc heard with the stethoscope 89 while viewing the cuff pressure Pc displayed in real time as the bar graph 50b on the indicator 50, and recorded the cuff pressure at that time (this recorded value is called the “HOLD value”). Then, the difference (HOLD value−auscultatory value) between the obtained HOLD value and the auscultatory value was obtained for each of 60 measurements. FIG. 5A shows a scatter diagram where the auscultatory value is represented on the horizontal axis and the difference (HOLD value−auscultatory value) is represented on the vertical axis, for the systolic blood pressure value (maximum blood pressure). FIG. 5B shows a scatter diagram where the auscultatory value is represented on the horizontal axis and the difference (HOLD value−auscultatory value) is represented on the vertical axis, for the diastolic blood pressure value (minimum blood pressure). As seen from these figures, the mean value of the differences (HOLD value−auscultatory value) for the systolic blood pressure value (maximum blood pressure) was −1.5 mmHg. On the other hand, the mean value of the differences (HOLD value−auscultatory value) for the diastolic blood pressure value (minimum blood pressure) was −1.2 mmHg. In this manner, regarding the systolic blood pressure value (maximum blood pressure), no significant difference was found in this validation experiment with respect to the conventional examples. However, it can be said that for the diastolic blood pressure value (minimum blood pressure), the phenomenon of being evaluated lower by about one pulse beat equivalent can be corrected.

Note that the sphygmomanometer 1A described above is configured to include the manual pump 32 and the manual valve 33 as the cuff pressure adjustment unit, and adjustment of pressurizing or depressurizing the cuff pressure Pc is manually performed. However, the present invention is not limited to this. For example, as in a commercially available electronic sphygmomanometer (e.g., an upper arm type sphygmomanometer HEM-7120 series manufactured by OMRON Healthcare Co., Ltd), an electric pump and a valve may be included, and the control unit 110 may perform control to automatically pressurize or depressurize the cuff pressure Pc. In such a case, the observer 80 no longer needs to operate the manual pump 32 and the manual valve 33, and one hand (in the example of FIG. 2, the right hand) can be used exclusively to press the HOLD button 52B during the depressurization process. Therefore, it is convenient for the observer 80. This point is similar in the second to fourth embodiments described below.

### Second Embodiment

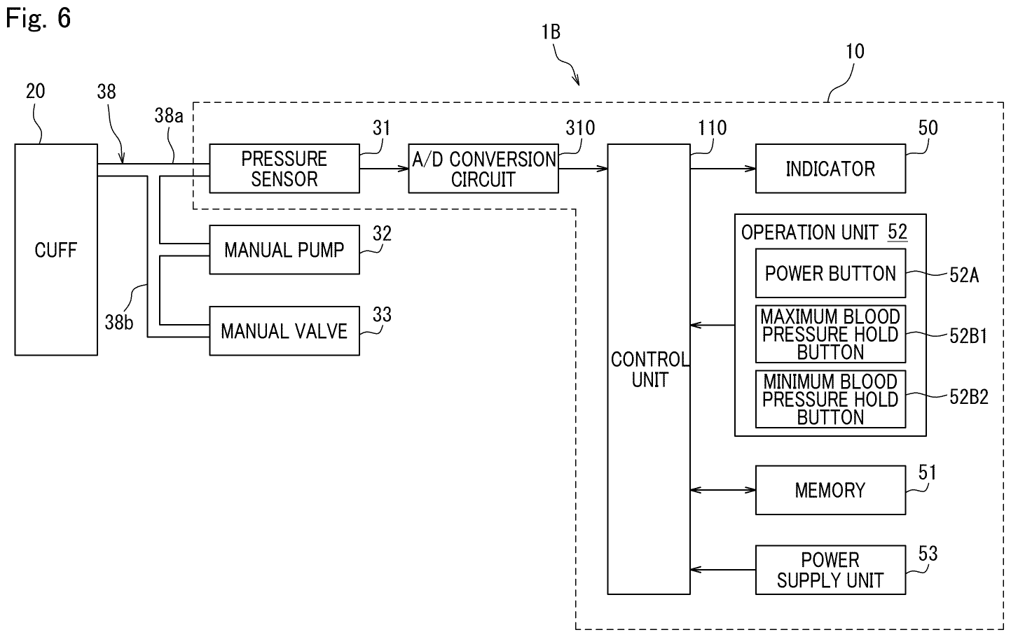

FIG. 6 shows a block configuration of a sphygmomanometer 1B as the second embodiment of the blood pressure recording device of this invention. This sphygmomanometer 1B is different from the sphygmomanometer 1A described above in including, as the HOLD button of the operation unit 52, a maximum blood pressure HOLD button 52B1 as a first switch that receives an instruction for recording a systolic blood pressure value and a minimum blood pressure HOLD button 52B2 as a second switch that receives an instruction for recording a diastolic blood pressure value. In this example, both the maximum blood pressure HOLD button 52B1 and the minimum blood pressure HOLD button 52B2 are momentary type (self-return type) switches. The hardware configurations of this sphygmomanometer 1B other than the operation unit 52 are the same as those of the sphygmomanometer 1A. In FIG. 6, the same components as those in FIG. 1 are denoted by the same reference signs, and redundant description will be omitted.

In this example, the observer 80 is scheduled to press the maximum blood pressure HOLD button 52B1 at a time when he judges that the Korotkoff sound Kc has started generating. The observer 80 is scheduled to press the minimum blood pressure HOLD button 52B2 at a time when he judges that the Korotkoff sound Kc has stopped generating.

FIG. 7 shows a flow of a blood pressure recording method of an embodiment in which the observer 80 measures and records the blood pressure of the upper arm 91 of the subject 90 using the sphygmomanometer 1B. As preparation for blood pressure measurement, similarly to FIG. 2, this example assumes that the observer 80 puts the cuff 20 on around the upper arm 91 of the subject 90, and brings the stethoscope 89 into contact with a part of the upper arm 91 of the subject 90 where the artery passes (a part corresponding to downstream of the cuff 20). The manual valve 33 is assumed to be opened.

Steps S201 to S209 of the flow of the blood pressure recording method of FIG. 7 are performed exactly similarly to steps S101 to S109 of the flow of the blood pressure recording method of FIG. 3.

In this example, while the processing of steps S208 and S209 of FIG. 7 is performed in the depressurization process, the control unit 110 determines in step S210 whether or not the maximum blood pressure HOLD button 52B1 is pressed. In step S213, the control unit 110 determines whether or not the minimum blood pressure HOLD button 52B2 is pressed. When neither the maximum blood pressure HOLD button 52B1 nor the minimum blood pressure HOLD button 52B2 has been pressed (No in step S210, No in step S213), the control unit 110 continues the processing of steps S208 to S210 and S213 as long as the current cuff pressure Pc is higher than a predetermined pressure (in this example, 30 mmHg) (No in step S216).

During the depressurization process, when the observer 80 presses the maximum blood pressure HOLD button 52B1 as shown in step S211 of FIG. 7 (Yes in step S210 of FIG. 7), the control unit 110 determines in step S212 that the first timing signal t1 was input at a time t1 at which the maximum blood pressure HOLD button 52B1 was pressed as exemplified in the middle part of FIG. 4. Then, the control unit 110 acts as a recording processing unit, and specifies a data pair (in the example in the lower part of FIG. 4, the data pair with the pulse beat number N=3 stored immediately before the time t1) corresponding to the time indicated by the first timing signal t1 among the data set DS1 stored in the memory 51. Furthermore, the control unit 110 causes the memory 51 to store, as a systolic blood pressure value, the cuff pressure Pc included in the specified data pair. In the example in the lower part of FIG. 4, 135 mmHg (indicated by the broken line frame A1) included in the data pair with the pulse beat number N=3 is stored as the systolic blood pressure value. Together with this, in this example, the obtained systolic blood pressure value 135 mmHg is displayed on the indicator 50. This display may be displayed as a digital value on the indicator 50 as, for example, “Maximum Blood Pressure 135 mmHg”, and/or may be displayed as a horizontal bar-shaped mark indicating a pressure value by a position along the scale 50a, such as a mark M1 shown in FIG. 10 described later.

On the other hand, during the depressurization process, when the observer 80 presses the minimum blood pressure HOLD button 52B2 as shown in step S214 of FIG. 7 (Yes in step S213 of FIG. 7), the control unit 110 determines in step S215 that the second timing signal t2 was input at a time t2 when the minimum blood pressure HOLD button 52B2 was pressed as exemplified in the middle part of FIG. 4. Then, the control unit 110 acts as the recording processing unit, and specifies a data pair (in the example in the lower part of FIG. 4, the data pair with the pulse beat number N=18 stored immediately before the time t2) corresponding to the time indicated by the second timing signal t2 among the data set DS1 stored in the memory 51, and further specifies a data pair (in the example in the lower part of FIG. 4, the data pair with the pulse beat number N=17) corresponding to one pulse beat before the data pair. Furthermore, the control unit 110 causes the memory 51 to store, as a corrected diastolic blood pressure value, the cuff pressure Pc included in the latter data pair. In the example in the lower part of FIG. 4, 95 mmHg (indicated by the broken line frame A2) included in the data pair with the pulse beat number N=17 is stored as the corrected diastolic blood pressure value. Together with this, in this example, the control unit 110 acts as a third display processing unit, and causes the indicator 50 to display the corrected diastolic blood pressure value 95 mmHg. This display may be displayed as a digital value on the indicator 50 as, for example, “Minimum Blood Pressure 95 mmHg”, and/or may be displayed as a horizontal bar-shaped mark indicating a pressure value by a position along the scale 50a, such as a mark M2 shown in FIG. 10 described later. This enables the observer 80 to easily recognize the systolic blood pressure value and the corrected diastolic blood pressure value obtained above by viewing the indicator 50.

Thereafter, when the depressurization process proceeds and the current cuff pressure Pc becomes a predetermined pressure (in this example, 30 mmHg) or less (Yes in step S216 of FIG. 7), the control unit 110 ends the processing of the flow of this blood pressure recording method. The observer 80 opens the manual valve 33 to decrease the cuff pressure Pc (step S217). Note that the observer 80 may open the manual valve 33 to decrease the cuff pressure Pc as soon as the corrected diastolic blood pressure value is recorded.

According to this second embodiment, similarly to the first embodiment, the systolic blood pressure value and the diastolic blood pressure value (corrected diastolic blood pressure value) can be accurately recorded. Moreover, according to this second embodiment, both the systolic blood pressure value and the corrected diastolic blood pressure value can be quickly recorded according to the intention of the observer 80 (whether to record the systolic blood pressure value or to record the diastolic blood pressure value). The observer 80 can end the blood pressure measurement as soon as the corrected diastolic blood pressure value can be recorded even in the middle of the depressurization process. Moreover, since the control unit 110 does not need to determine whether the operation of the operation unit 52 is the first operation or the second operation in the depressurization process, the processing by the control unit 110 is simplified.

### Third Embodiment

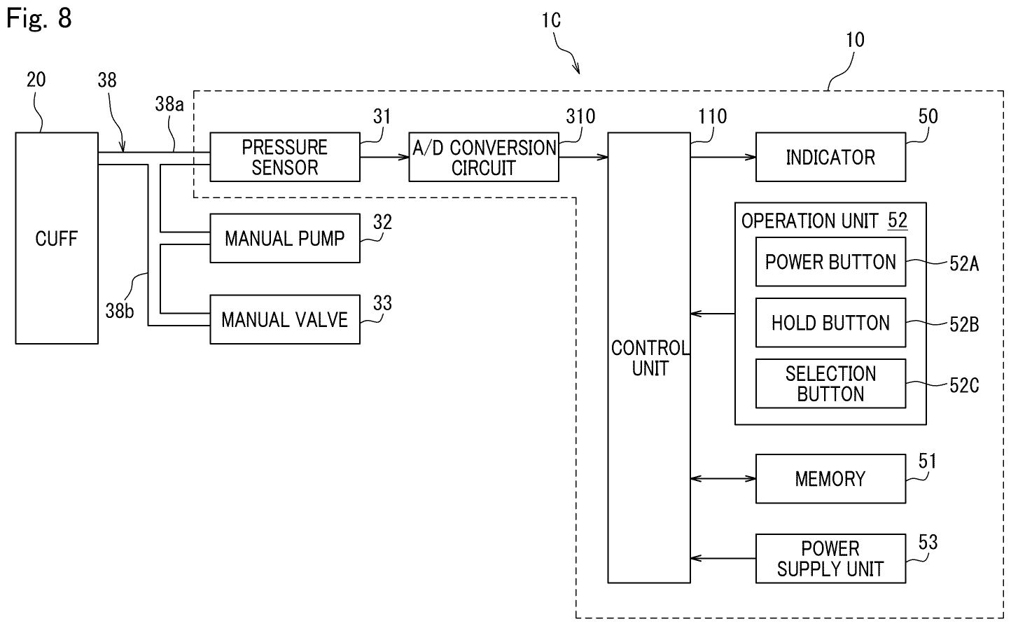

FIG. 8 shows a block configuration of a sphygmomanometer 1C as the third embodiment of the blood pressure recording device of this invention. This sphygmomanometer 1C is different from the sphygmomanometer 1A described above in including, as the operation unit 52, a selection button 52C as a selection operation unit in addition to the power button 52A and the HOLD button 52B. In this example, the selection button 52C is a momentary type (self-return type) switch. The hardware configurations of this sphygmomanometer 1C other than the operation unit 52 are the same as those of the sphygmomanometer 1A. In FIG. 8, the same components as those in FIG. 1 are denoted by the same reference signs, and redundant description will be omitted.

In this example, the observer 80 is scheduled to select an option that, the observer himself/herself considers, corresponds to the diastolic blood pressure value with the selection button 52C from the options (the cuff pressure Pc included in the data pair) (i.e., marks M1 and M2 in FIG. 10 described later) corresponding to the times at which the first and second timing signals t1 and t2 were input, displayed on the indicator 50.

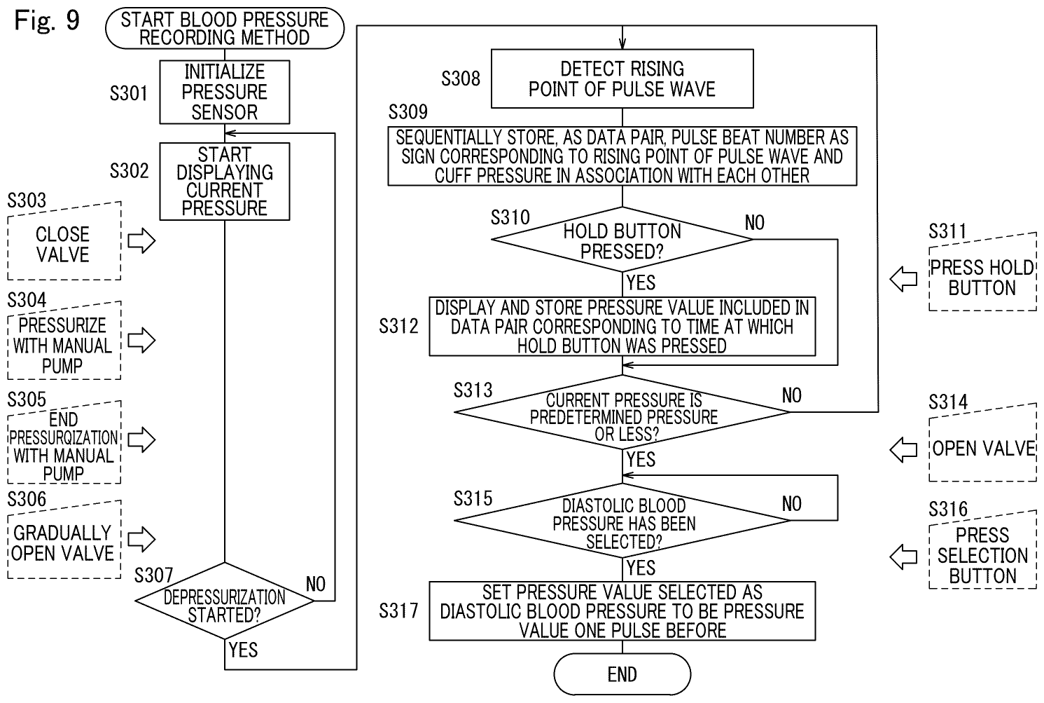

FIG. 9 shows a flow of a blood pressure recording method of an embodiment in which the observer 80 measures and records the blood pressure of the upper arm 91 of the subject 90 using the sphygmomanometer 1C. As preparation for blood pressure measurement, similarly to FIG. 2, this example assumes that the observer 80 puts the cuff 20 on around the upper arm 91 of the subject 90, and brings the stethoscope 89 into contact with a part of the upper arm 91 of the subject 90 where the artery passes (a part corresponding to downstream of the cuff 20). The manual valve 33 is assumed to be opened.

Steps S301 to S309 of the flow of the blood pressure recording method of FIG. 9 are performed exactly similarly to steps S101 to S109 of the flow of the blood pressure recording method of FIG. 3.

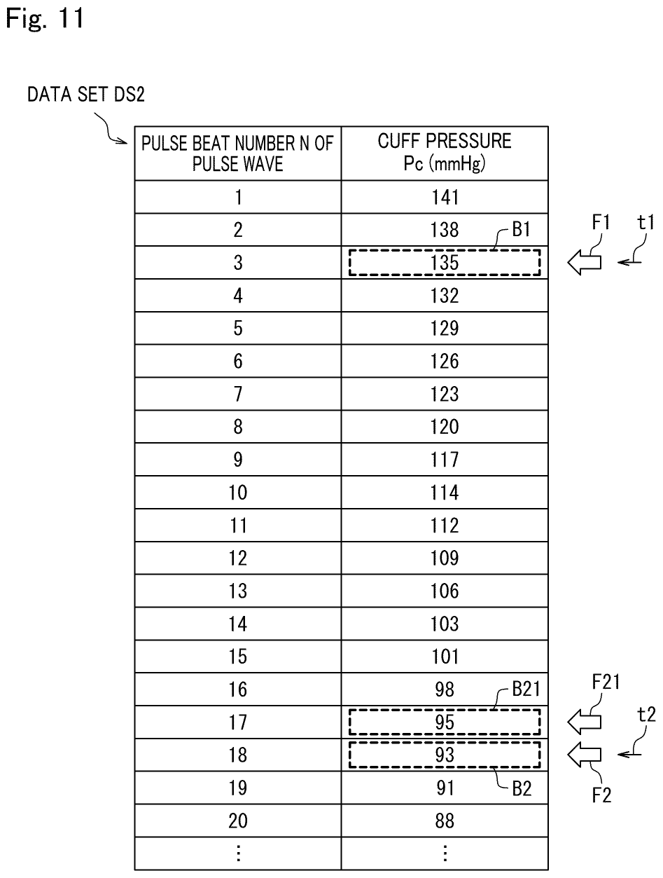

In this example, while the processing of steps S308 and S309 of FIG. 9 is performed in the depressurization process, the control unit 110 determines in step S310 whether or not the HOLD button 52B is pressed. When the HOLD button 52B is not pressed (No in step S310), the control unit 110 continues the processing of steps S308 to S310 as long as the current cuff pressure Pc is higher than a predetermined pressure (in this example, 30 mmHg) (No in step S313). In this example, a data set DS2 shown in FIG. 11 is assumed to be stored in the memory 51.

During the depressurization process, when the observer 80 presses the HOLD button 52B for the first time as shown in step S311 of FIG. 9 (Yes in step S310 of FIG. 9), in step S312, the control unit 110 acts as a data pair specification unit and specifies, among the data set DS2 stored in the memory 51, a data pair (in the example of FIG. 11, the data pair with the pulse beat number N=3) corresponding to the time t1 at which the HOLD button 52B was pressed. Furthermore, the control unit 110 sets a flag F1 to the specified data pair with the pulse beat number N=3, thereby maintaining information indicating the specified data pair with the pulse beat number N=3. Together with this, the control unit 110 acts as a second display processing unit and causes the indicator 50 to display, as an option of the blood pressure value, the cuff pressure Pc (in the example of FIG. 11, 135 mmHg indicated by a broken line frame B1) included in the specified data pair with the pulse beat number N=3. In this example, as the mark M1 shown in FIG. 10, portion (A), a horizontal bar-shaped mark indicating the pressure value by the position along the scale 50a is displayed. Note that at this time point, the data set DS2 only stores the data pair with the pulse beat number N=1 to the data pair with the pulse beat number N=3.

Subsequently, during the depressurization process, when the observer 80 presses the HOLD button 52B for the second time as shown in step S311 of FIG. 9 (Yes in step S310 of FIG. 9), again in step S312, the control unit 110 acts as the data pair specification unit and specifies, among the data set DS2 stored in the memory 51, a data pair (in the example of FIG. 11, the data pair with the pulse beat number N=18) corresponding to the time t2 at which the HOLD button 52B was pressed. Furthermore, the control unit 110 sets a flag F2 to the specified data pair with the pulse beat number N=18, thereby maintaining information indicating the specified data pair with the pulse beat number N=18. Together with this, the control unit 110 acts as the second display processing unit and causes the indicator 50 to display, as an option of the blood pressure value, the cuff pressure Pc (in the example of FIG. 11, 93 mmHg indicated by a broken line frame B2) included in the specified data pair with the pulse beat number N=18. In this example, as the mark M2 shown in FIG. 10, portion (A), a horizontal bar-shaped mark indicating the pressure value by the position along the scale 50a is displayed.

Note that if the HOLD button 52B is pressed three times or more during the depressurization process, the control unit 110 acts as the data pair specification unit and specifies, among the data set DS1 stored in the memory 51, a data pair corresponding to the time at which the HOLD button 52B was pressed. Furthermore, the control unit 110 sets a flag to the specified data pair with the pulse beat number N, thereby maintaining information indicating the specified data pair with the pulse beat number N. Together with this, the control unit 110 acts as the second display processing unit and causes the indicator 50 to display, as an option of the blood pressure value, the cuff pressure Pc included in the specified data pair with the pulse beat number N. Note that the “three times or more” is configured to be able to cope with the possibility that the HOLD button 52B is pressed three times or more during the depressurization process. Specifically, if the subject 90 exhibits arrhythmias, the pulse beat may be interrupted midway. In a case of a pregnant woman, the Korotkoff sound may continue to be heard down to a low pressure (e.g., 30 mmHg). The sound quality of the Korotkoff sound varies in five stages from generation to stop. Each stage of sound is called a “phase”, and there are phase I to phase V. The point (beginning of phase IV) at which the sound quality of the Korotkoff sound changes and the last sound of the Korotkoff sound may be both recorded. Therefore, as described above, it is configured to be able to cope with the possibility that the HOLD button 52B is pressed three times or more during the depressurization process.

Thereafter, when the depressurization process proceeds and the current cuff pressure Pc becomes a predetermined pressure (in this example, 30 mmHg) or less (Yes in step S313 of FIG. 9), the observer 80 opens the manual valve 33 to reduce the cuff pressure Pc, and ends the depressurization process (step S314). At this stage of the depressurization process ending, the control unit 110 acts as the second display processing unit, and maintains the display of the mark M1 representing the obtained blood pressure value of 135 mmHg and the display of the mark M2 representing the obtained blood pressure value of 93 mmHg as exemplified in FIG. 10, portion (A). Then, the control unit 110 waits for an option to be selected with the selection button 52C (step S315 of FIG. 9). At this stage, the observer 80 selects an option that, the observer himself/herself considers, corresponds to the diastolic blood pressure value with the selection button 52C from the marks M1 and M2 as the options corresponding to the times (i.e., the times at which the timing signals were input) t1 and t2 when the HOLD button 52B was pressed displayed on the indicator 50.

In this example, every time the observer 80 presses the selection button 52C once, the mark M1 blinks as shown in FIG. 10, portion (B) (indicated by a radial mark surrounding the horizontal bar), or the mark M2 blinks as shown in FIG. 10, portion (C), and thus selection candidates are sequentially switched. Note that in a case where three or more marks as options are displayed, selection candidates are switched in a circulation manner among the three or more marks.

This example assumes that the observer 80 presses and holds the selection button 52C for 3 seconds or more, for example, in order to select the mark M2 in a state where the mark M2 blinks as shown in FIG. 10, portion (C). Then, in step S317 of FIG. 9, the control unit 110 acts as a recording processing unit, and specifies, as a data pair selected by the observer 80 as corresponding to the diastolic blood pressure value, a data pair (in the example of FIG. 11, among the data set DS2, the data pair with the pulse beat number N=18 to which the flag F2 is set) including the cuff pressure indicated by the option selected with the selection button 52C. Furthermore, the control unit 110 specifies a data pair (in the example of FIG. 11, among the data set DS2, the data pair with the pulse beat number N=17) corresponding to one pulse beat before the specified data pair. Then, the control unit 110 causes the memory 51 to store, as a corrected diastolic blood pressure value, the cuff pressure Pc included in the latter data pair. In this example, as shown in FIG. 11, information indicating that the corrected diastolic blood pressure value is 95 mmHg is stored by setting a flag F21 at 95 mmHg (indicated by a broken line frame B21) included in the data pair with the pulse beat number N=17 in the data set DS2. Together with this, in this example, the control unit 110 acts as a third display processing unit, and causes the indicator 50 to display the corrected diastolic blood pressure value 95 mmHg. In this example, in place of display of the mark M2, this display is displayed as a horizontal bar-shaped mark indicating the pressure value by the position along the scale 50a as in the mark M21 shown in FIG. 10, portion (D). In this example, the remaining mark M1 displays the cuff pressure Pc (in the example of FIG. 11, 135 mmHg indicated by the broken line frame B1) included in the data pair with the pulse beat number N=3 to which the flag F1 is set as it is. This enables the observer 80 to easily recognize the systolic blood pressure value and the corrected diastolic blood pressure value obtained above by viewing the mark M1 and the mark M21 displayed on the indicator 50. In this manner, the systolic blood pressure value and the corrected diastolic blood pressure value are recorded.

According to this third embodiment, similarly to the first embodiment, the systolic blood pressure value and the diastolic blood pressure value (corrected diastolic blood pressure value) can be accurately recorded. Moreover, according to this third embodiment, the corrected diastolic blood pressure value can be recorded according to the selection of the observer 80 (selection of which option corresponding to the diastolic blood pressure value).

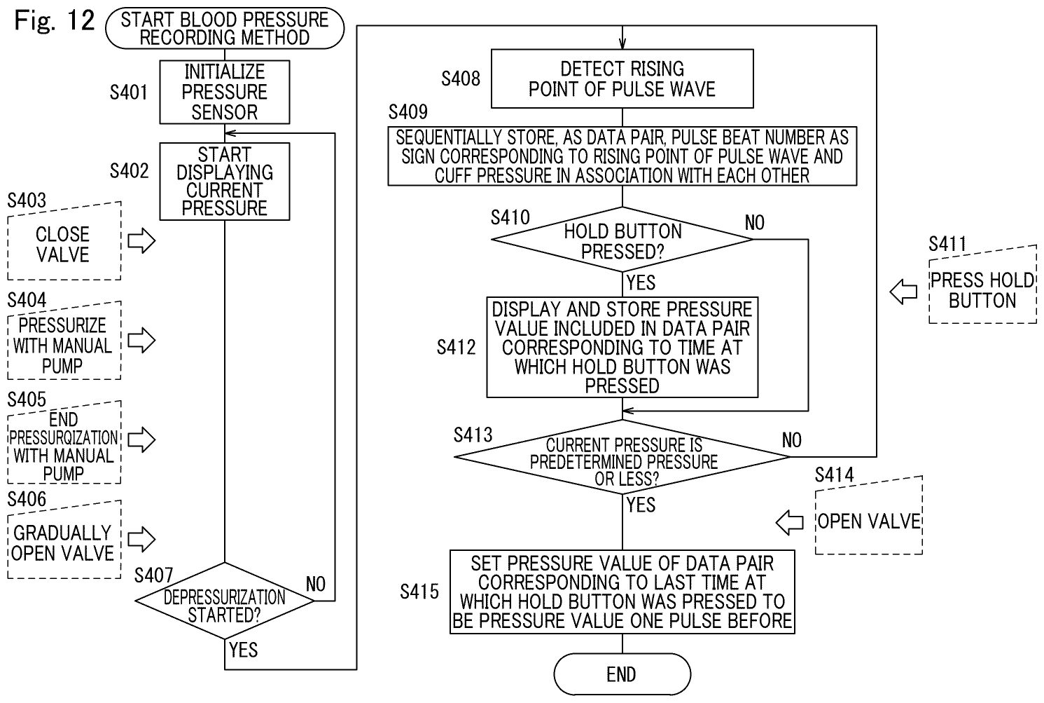

FIG. 12 shows a flow of a modification in which the flow of the blood pressure recording method of FIG. 9 is modified. As preparation for blood pressure measurement, similarly to FIG. 2, this example assumes that the observer 80 puts the cuff 20 on around the upper arm 91 of the subject 90, and brings the stethoscope 89 into contact with a part of the upper arm 91 of the subject 90 where the artery passes (a part corresponding to downstream of the cuff 20). The manual valve 33 is assumed to be opened.

Steps S401 to S414 of the flow of the blood pressure recording method of FIG. 12 are performed exactly similarly to steps S301 to S314 of the flow of the blood pressure recording method of FIG. 9.

In the flow of the blood pressure recording method of FIG. 12, when the HOLD button 52B is pressed twice or more in the depressurization process, in step S415 after the depressurization process ending, the control unit 110 acts as a recording processing unit, and uses, as the data pair corresponding to the time indicated by the second timing signal t2, the data pair (in the example of FIG. 11, the data pair with the pulse beat number N=18) corresponding to the time t2 at which the last operation was performed among the data pairs to which the flags (F1 and F2) are set in the data set DS2. That is, without requesting the observer 80 to select an option that, the observer 80 considers, corresponds to the diastolic blood pressure, the control unit 110 causes the memory 51 to store, as a corrected diastolic blood pressure value, the cuff pressure Pc included in the data pair corresponding to one pulse beat before the data pair with the pulse beat number N=18. In the example of FIG. 11, storage is performed by setting the flag F21 at 95 mmHg (indicated by the broken line frame B21) included in the data pair with the pulse beat number N=17 among the data set DS2. Together with this, in this example, the control unit 110 acts as a third display processing unit, and causes the indicator 50 to display the corrected diastolic blood pressure value 95 mmHg. In this example, in place of display of the mark M2, this display is displayed as a horizontal bar-shaped mark indicating the pressure value by the position along the scale 50a as in the mark M21 shown in FIG. 10, portion (D). In this example, the remaining mark M1 displays the cuff pressure Pc (in this example, 135 mmHg indicated by the broken line frame B1) included in the data pair with the pulse beat number N=3 to which the flag F1 is set as it is. This enables the observer 80 to easily recognize the systolic blood pressure value and the corrected diastolic blood pressure value obtained above by viewing the mark M1 and the mark M21 displayed on the indicator 50. In this manner, the systolic blood pressure value and the corrected diastolic blood pressure value are recorded.

According to the flow of the blood pressure recording method of FIG. 12, the processing by the control unit 110 acting as the recording processing unit is simplified. The selection button 52C can be omitted from the hardware configuration of the sphygmomanometer 1C.

Note that in the first to third embodiments, with the start of the depressurization process, the control unit 110 acts as the first data accumulation unit, and starts the processing of causing the memory 51 to sequentially store, as a data pair, the pulse beat number N as a sign corresponding to the time at which the rising point Pf of the pulse beat was generated and the cuff pressure Pc at the time when the rising point Pf of the pulse beat was generated, in association with each other. However, the present invention is not limited to this. The control unit 110 may be configured to start causing the memory 51 to sequentially store the data pair, during the depressurization process, from the data pair (in the example of FIG. 11, the data pair with the pulse beat number N=3) corresponding to the time at which the HOLD button 52B was pressed for the first time. This can save the resources of the memory 51d.

### Fourth Embodiment

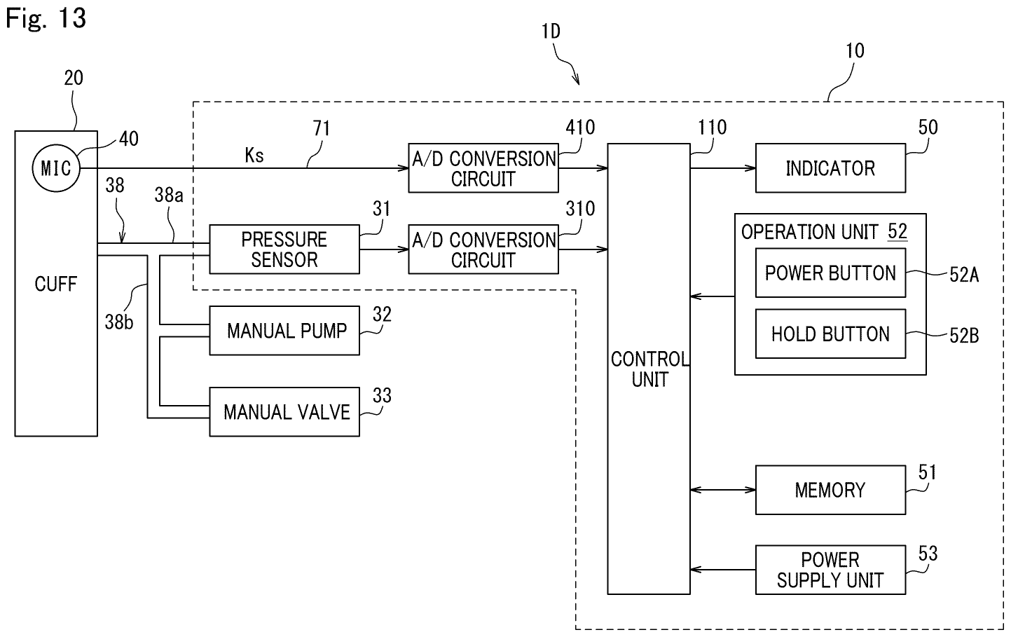

FIG. 13 shows a block configuration of a sphygmomanometer 1D as the fourth embodiment of the blood pressure recording device of this invention. This sphygmomanometer 1D is different from the sphygmomanometer 1A described above in including a microphone 40 mounted on the cuff 20 and an A/D conversion circuit 410 mounted on the main body 10. The microphone 40 converts a sound generated by the artery through the upper arm 91 of the subject 90 into a sound signal Ks that is an electric signal and outputs the sound signal Ks. The sound signal Ks output from the microphone 40 is transmitted to the A/D conversion circuit 410 through a wire 71 extending from the cuff 20 to the main body 10 shown in FIG. 13. The A/D conversion circuit 410 converts the sound signal Ks from an analog value into a digital value, and transmits the latter to the control unit 110. Other hardware configurations of this sphygmomanometer 1D are the same as those of the sphygmomanometer 1A. In FIG. 13, the same components as those in FIG. 1 are denoted by the same reference signs, and redundant description will be omitted.

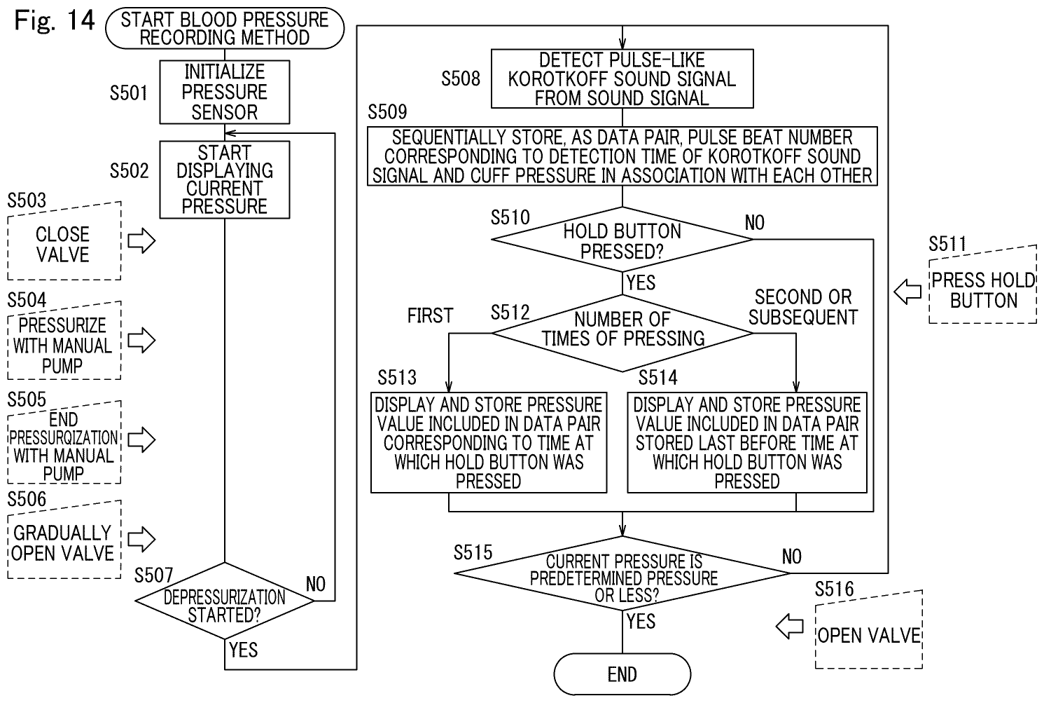

FIG. 14 shows a flow of a blood pressure recording method of an embodiment in which the observer 80 measures and records the blood pressure of the upper arm 91 of the subject 90 using the sphygmomanometer 1D. As preparation for blood pressure measurement, similarly to FIG. 2, this example assumes that the observer 80 puts the cuff 20 on around the upper arm 91 of the subject 90, and brings the stethoscope 89 into contact with a part of the upper arm 91 of the subject 90 where the artery passes (a part corresponding to downstream of the cuff 20). The manual valve 33 is assumed to be opened.

Steps S501 to S507 of the flow of the blood pressure recording method of FIG. 14 are performed exactly similarly to steps S101 to S107 of the flow of the blood pressure recording method of FIG. 3.

When the depressurization is started (Yes in step S507), the control unit 110 performs the processing of steps S508 to S514 in this depressurization process.

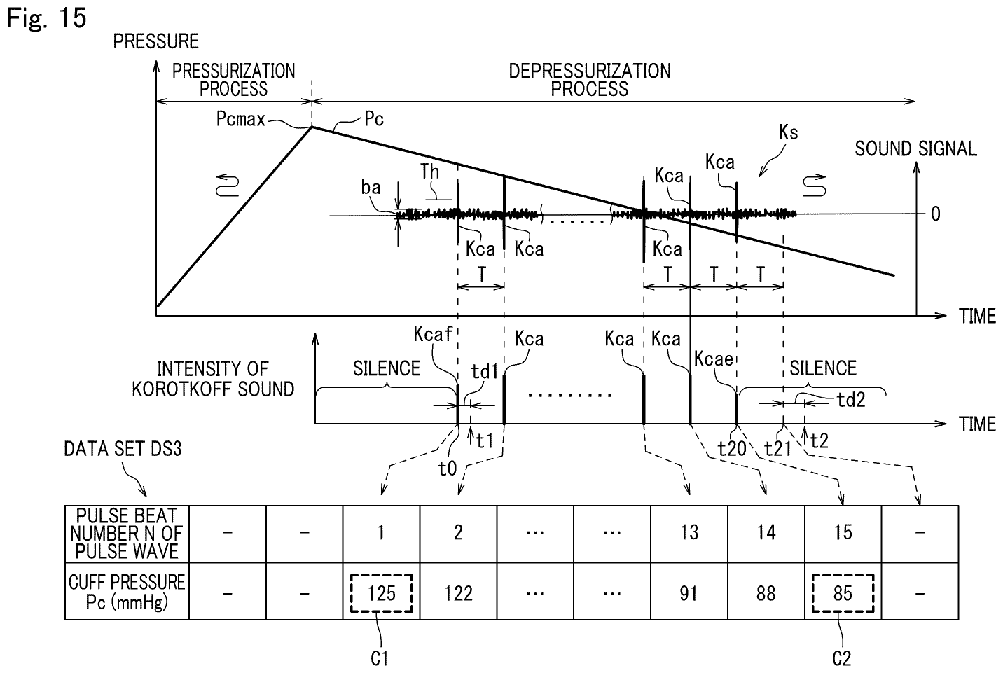

First, the microphone 40 converts a sound generated by the artery through the upper arm 91 of the subject 90 into the sound signal Ks that is an electric signal and outputs the sound signal Ks. As shown in the upper part of FIG. 15, this sound signal Ks includes a pulse-like Korotkoff sound signal Kca indicating a Korotkoff sound as an electric signal (alternating-current signal). The sound signal Ks output from the microphone 40 is transmitted to the control unit 110 as a digital value via the wire 71 and the A/D conversion circuit 410 shown in FIG. 13. Here, in step S508 of FIG. 14, the control unit 110 acts as a Korotkoff sound signal detection unit, and detects the Korotkoff sound signal Kca from the sound signal Ks, for example, as shown in the middle part of FIG. 15. As shown in the upper part of FIG. 15, for example, the control unit 110 sets a threshold Th slightly higher than a background noise level ba for the sound signal Ks and extracts a component exceeding the threshold Th from the sound signal Ks, whereby the Korotkoff sound signal Kca can be detected. It is assumed that the timing at which the Korotkoff sound signal Kca is detected coincides with the timing at which the observer 80 listens to the Korotkoff sound Kc with the stethoscope 89 (i.e., the processing time for the control unit 110 to detect the Korotkoff sound signal Kca from the sound signal Ks is assumed to be negligible).

Concurrently with this processing, in step S509 of FIG. 14, the control unit 110 acts as a second data accumulation unit, and starts processing of causing the memory 51 to sequentially store, as a data pair, a pulse beat number N as a sign corresponding to the time at which the Korotkoff sound signal Kca was detected and the cuff pressure Pc at the time when the Korotkoff sound signal Kca was detected, in association with each other. In this example, as shown in the lower part of FIG. 15, as a series of data pairs (this is called a “data set”) DS3 obtained in this depressurization process, a data pair with the pulse beat number N=1 and the cuff pressure Pc=125 (mmHg) is stored, then a data pair with the pulse beat number N=2 and the cuff pressure Pc=122 (mmHg) is stored, and in this manner, a data pair with the pulse beat number N=15 and the cuff pressure Pc=85 (mmHg) is finally stored. Since this processing is performed without depending on the operation of the observer 80, this data set DS3 is stored without being affected by the reaction time (described later) when the observer 80 presses the HOLD button 52B. Note that in the lower part of FIG. 15, there is no data pair in the column denoted by “-”.

Note that the time at which the Korotkoff sound signal Kca was generated and the cuff pressure Pc at the time when the Korotkoff sound signal Kca was generated may be directly associated with each other and sequentially stored in the memory 51 as a data pair. However, use of the pulse beat number N as described above enables resources of the memory 51 to be saved. This point is similar to that in the first embodiment.

While the processing in steps S508 and S509 of FIG. 14 is being performed in the depressurization process, the control unit 110 determines in step S510 whether or not the HOLD button 52B is pressed. When the HOLD button 52B is not pressed (No in step S510), the control unit 110 continues the processing of steps S508 to S510 as long as the current cuff pressure Pc is higher than a predetermined pressure (in this example, 30 mmHg) (No in step S515). Note that the 30 mmHg described above is a pressure lower than an assumed minimum blood pressure, and is determined in advance as a pressure at which the depressurization process should be ended.

During the depressurization process, the observer 80 listens to the Korotkoff sound Kc generated by the artery through the upper arm 91 with the stethoscope 89, similarly to the first embodiment in this example, while viewing the cuff pressure Pc displayed in real time as the bar graph 50b on the indicator 50 shown in FIG. 2 (as described above, it is assumed that the timing at which the Korotkoff sound Kc was generated coincides with the timing at which the Korotkoff sound signal Kca shown in the middle part of FIG. 15 was generated). Then, in step S511 of FIG. 14, the observer 80 presses the HOLD button 52 at a time t1 delayed by a reaction time td1 from a time t0 at which he could judge that the Korotkoff sound Kc had started generating (i.e., a time at which he heard the first Korotkoff sound Kcaf from a silent state) as exemplified in the middle part of FIG. 15. This corresponds to the first pressing in the depressurization process. Note that it is assumed that the reaction time td1 of the observer 80 normally does not exceed 1 pulse beat T.

When the HOLD button 52B is pressed (Yes in step S510 of FIG. 14), the control unit 110 acts as a recording processing unit, and determines whether a number of times of pressing of the HOLD button 52B in the depressurization process is a first time or a second or subsequent time in step S512 of FIG. 14. In this example, by referring to the count value of the counter described in the first embodiment, the control unit 110 can determine whether the number of times of pressing of the HOLD button 52B is the first time or the second or subsequent time. Here, if the number of times of pressing of the HOLD button 52B is the first time, in step S513, the control unit 110 determines that a first timing signal t1 (for simplicity, represented by the same reference sign as the time t1) was input as exemplified in the middle part of FIG. 15. Then, the control unit 110 acts as the recording processing unit, and specifies a data pair (in the example in the lower part of FIG. 15, the data pair with the pulse beat number N=1 stored immediately before the time t1) corresponding to the time indicated by the first timing signal t1 among the data set DS3 stored in the memory 51. Furthermore, the control unit 110 causes the memory 51 to store, as a systolic blood pressure value, the cuff pressure Pc included in the specified data pair. In the example in the lower part of FIG. 15, 125 mmHg (indicated by a broken line frame C1) included in the data pair with the pulse beat number N=1 is stored as the systolic blood pressure value. Together with this, in this example, the obtained systolic blood pressure value 125 mmHg is displayed on the indicator 50. This display may be displayed as a digital value on the indicator 50 as, for example, “Maximum Blood Pressure 125 mmHg”, and/or may be displayed as a horizontal bar-shaped mark indicating a pressure value by a position along the scale 50a, such as the mark M1 shown in FIG. 10. This enables the observer 80 to easily recognize the obtained systolic blood pressure value by viewing the indicator 50. Note that at this time point, the data set DS3 only stores the data pair with the pulse beat number N=1.

Thereafter, the depressurization process proceeds, and the cuff pressure Pc decreases. In step S511 of FIG. 14 again, the observer 80 presses the HOLD button 52 at a time t2 delayed by a reaction time td2 from a time t21 at which he could judge that the Korotkoff sound Kc had stopped generating (i.e., a time at which he could judge for the first time that he no longer heard any Korotkoff sound after being delayed by 1 pulse beat T from a time t20 at which he could hear the last Korotkoff sound Kcae) as exemplified in the middle part of FIG. 15. This corresponds to the second pressing in the depressurization process. Note that it is assumed that the reaction time td2 of the observer 80 normally does not exceed 1 pulse beat T.

When the HOLD button 52B is pressed (Yes in step S510 of FIG. 14), the control unit 110 determines whether the number of times of pressing of the HOLD button 52B in the depressurization process is the first time or the second or subsequent time in step S512 of FIG. 14. Here, by referring to the count value of the counter, if the number of times of pressing of the HOLD button 52B is the second time, in step S514, the control unit 110 determines that a second timing signal t2 (for simplicity, represented by the same reference sign as the time t2) was input as exemplified in the middle part of FIG. 15. Then, the control unit 110 acts as the recording processing unit, specifies a data pair (in the example in the lower part of FIG. 15, a data pair with the pulse beat number N=15) stored last before the time indicated by the second timing signal t2 among the data set DS3 stored in the memory 51, and causes the memory 51 to store, as a corrected diastolic blood pressure value, the cuff pressure Pc included in the specified data pair. In the example in the lower part of FIG. 15, 85 mmHg (indicated by a broken line frame C2) included in the data pair with the pulse beat number N=15 is stored as the corrected diastolic blood pressure value. Together with this, in this example, the control unit 110 acts as a third display processing unit, and causes the indicator 50 to display the corrected diastolic blood pressure value 85 mmHg. This display may be displayed as a digital value on the indicator 50 as, for example, “Minimum Blood Pressure 85 mmHg”, and/or may be displayed as a horizontal bar-shaped mark indicating a pressure value by a position along the scale 50a, such as the mark M2 shown in FIG. 10. This enables the observer 80 to easily recognize the corrected diastolic blood pressure value by viewing the indicator 50.

Note that the second “or subsequent” in step S512 of FIG. 14 is configured to be able to cope with the possibility that the HOLD button 52B is pressed three times or more during the depressurization process. Specifically, if the subject 90 exhibits arrhythmias, the pulse beat may be interrupted midway. In a case of a pregnant woman, the Korotkoff sound may continue to be heard down to a low pressure (e.g., 30 mmHg). The sound quality of the Korotkoff sound varies in five stages from generation to stop. Each stage of sound is called a “phase”, and there are phase I to phase V. The point (beginning of phase IV) at which the sound quality of the Korotkoff sound changes and the last sound of the Korotkoff sound may be both recorded. Therefore, as described above, it is configured to be able to cope with the possibility that the HOLD button 52B is pressed three times or more during the depressurization process.

Thereafter, when the depressurization process proceeds and the current cuff pressure Pc becomes a predetermined pressure (in this example, 30 mmHg) or less (Yes in step S515), the control unit 110 ends the processing of the flow of this blood pressure recording method. The observer 80 opens the manual valve 33 to decrease the cuff pressure Pc (step S516). Note that the observer 80 may open the manual valve 33 to decrease the cuff pressure Pc as soon as the corrected diastolic blood pressure value is recorded.

In this manner, in this sphygmomanometer 1D, the control unit 110 acts as the recording processing unit, specifies the data pair stored last before the time indicated by the second timing signal t2 among the series of data pairs stored in the memory 51 in the depressurization process, and records, as the corrected diastolic blood pressure value, the cuff pressure Pc included in the specified data pair. According to the analysis of the present inventor described above, the corrected diastolic blood pressure value corresponds to not the time t21 at which the observer 80 can judge for the first time that he no longer hears the Korotkoff sound Kc from a state where he continued to hear the Korotkoff sound Kc for each pulse beat but the cuff pressure Pc corresponding to the time t20 at which he actually heard the last Korotkoff sound Kc. Therefore, according to the flow of the blood pressure recording method in FIG. 14 using the sphygmomanometer 1D, it is possible to correct a phenomenon that the minimum blood pressure is evaluated lower by about one pulse beat equivalent.

It is considered that some reaction times td1 and td2, respectively, are required from when the observer judged that the Korotkoff sound Kc had started generating to when he presses the HOLD button 52B, and from when the observer 80 judged that the Korotkoff sound had stopped generating to when he presses the HOLD button 52B. However, in this sphygmomanometer 1D, the control unit 110 acts as the second data accumulation unit in the depressurization process, whereby data pairs are sequentially stored in the memory 51 without being affected by the reaction times td1 and td2. Therefore, the data pair specified by the control unit 110 acting as the recording processing unit is stored without being affected by the reaction times td1 and td2 of the observer 80. As a result, the systolic blood pressure value and the corrected diastolic blood pressure value recorded above are not actually affected by the reaction times td1 and td2 of the observer 80.

As described above, according to this fourth embodiment, the systolic blood pressure value and the diastolic blood pressure value (corrected diastolic blood pressure value) can be accurately recorded as compared with the conventional examples.

In this fourth embodiment, in the data set DS3, the memory 51 is caused to sequentially store, as the data pair, the pulse beat number N as the sign corresponding to the time at which the Korotkoff sound signal Kca was detected and the cuff pressure Pc at the time when the Korotkoff sound signal Kca was detected, in association with each other. Therefore, as compared with the first embodiment, there is no possibility that a deviation (time interval) between the time at which the rising point Pf of the pulse beat is generated and the time at which the Korotkoff sound Kc of the pulse beat is generated affects the accuracy of the blood pressure measurement (recording). Therefore, the systolic blood pressure value and the diastolic blood pressure value (corrected diastolic blood pressure value) can be more accurately recorded.

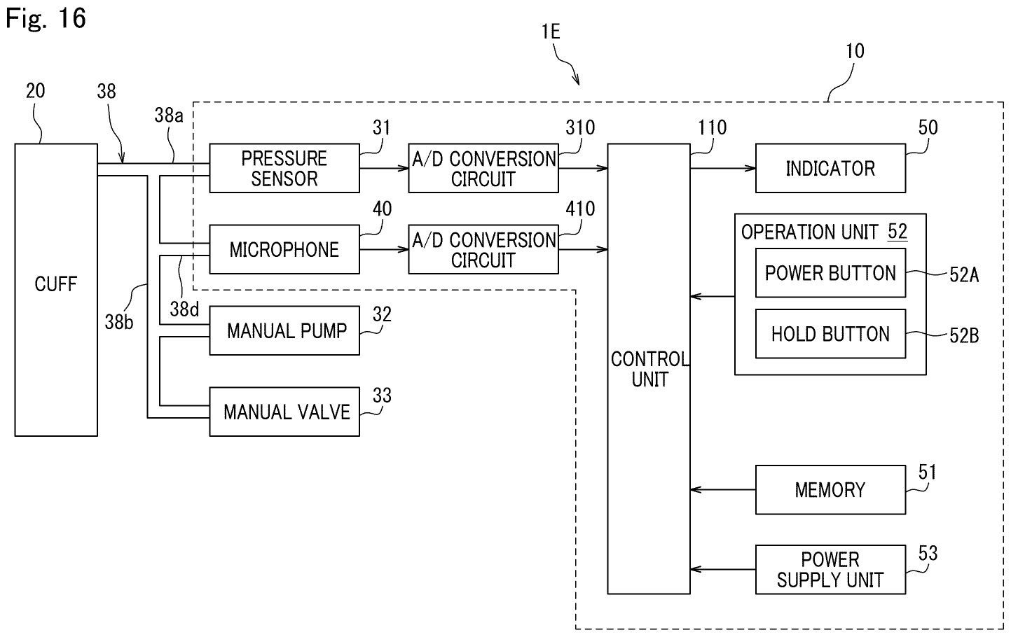

Note that in the sphygmomanometer 1D described above, the microphone 40 is mounted on the cuff 20, but the present invention is not limited to this. For example, as in a sphygmomanometer 1E shown in FIG. 16, the microphone 40 may be mounted on the main body 10, and may be configured to receive a sound generated by the artery through the upper arm 91 of the subject 90 through an air pipe 38d communicating with an air pipe 38 in this example. Also in this case, similarly to the sphygmomanometer 1D, the microphone 40 can convert a sound generated by the artery through the upper arm 91 of the subject 90 into a sound signal Ks that is an electric signal and output the sound signal Ks. Therefore, by executing the flow of the blood pressure recording method of FIG. 14 using this sphygmomanometer 1E, the systolic blood pressure value and the diastolic blood pressure value (corrected diastolic blood pressure value) can be accurately recorded. Moreover, when the sphygmomanometer 1E is used, it is not necessary to extend the wire 71 between the cuff 20 and the main body 10, and therefore the configuration of the device can be simplified.

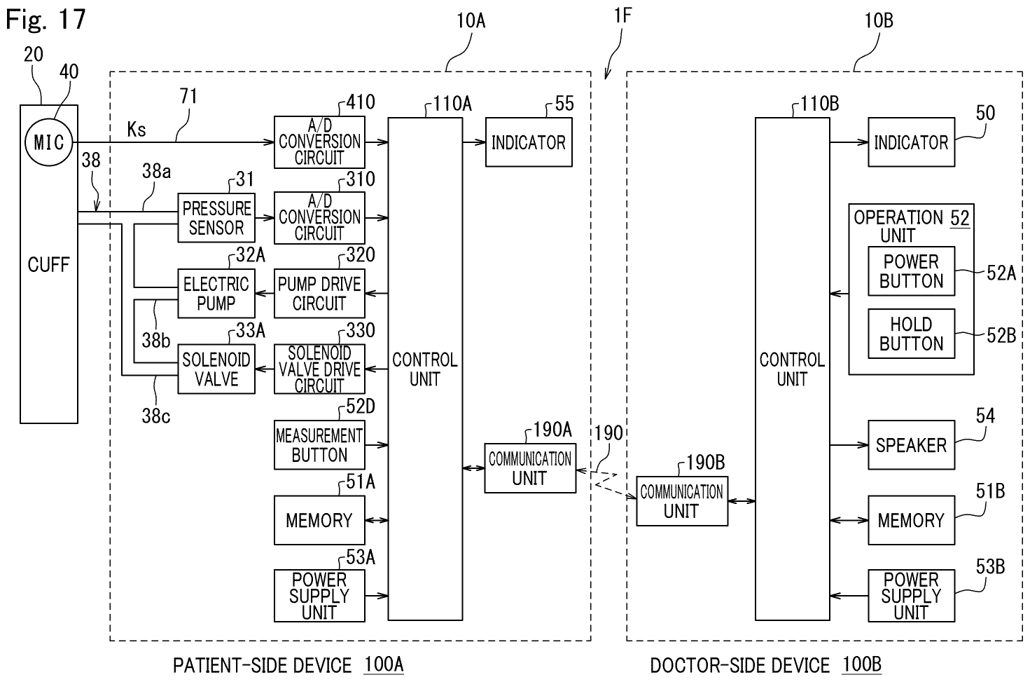

In this fourth embodiment, the observer 80 presses the HOLD button 52B by listening to the Korotkoff sound Kc generated by the artery through the upper arm 91 with the stethoscope 89 similarly to the first embodiment. However, the present invention is not limited to this. For example, it is assumed that an electro-acoustic conversion device such as a speaker 54 shown in FIG. 17 described later is provided, and the Korotkoff sound signal Kca is reproduced as the Korotkoff sound Kc by the electro-acoustic conversion device. In this case, the observer 80 may press the HOLD button 52B by listening to the reproduced Korotkoff sound Kc.

### Fifth Embodiment

FIG. 17 shows a block configuration of a blood pressure measurement system 1F as the fifth embodiment of the blood pressure recording device of this invention. This blood pressure measurement system 1F corresponds to a modification of the sphygmomanometer 1D of FIG. 13 to enable telemedicine. Note that in FIG. 17, the same components as those in FIG. 13 are denoted by the same reference signs, and redundant description will be omitted.

This blood pressure measurement system 1F roughly includes a patient-side device 100A provided in a location of the subject (in this example, a patient) 90 having the upper arm 91 and a doctor-side device 100B provided in a location of the observer (in this example, a medical doctor) 80. In this example, the location of the subject 90 refers to a location of a residence where the patient as the subject 90 lives. In this example, the location of the observer 80 refers to a location of a medical institution where the medical doctor as the observer 80 works.

The patient-side device 100 includes the cuff 20 and a main body 10A. The cuff 20 is mounted with the microphone 40. The main body 10A is mounted with a control unit 110A, the pressure sensor 31 as a pressure detection unit, an electric pump 32A and a solenoid valve 33A as a cuff pressure adjustment unit, a measurement button 52D as an operation unit, a memory 51A, a power supply unit 53A, the A/D conversion circuits 310 and 410, a pump drive circuit 320, a solenoid valve drive circuit 330, a communication unit 190A, and an indicator 55.

The sound signal Ks output from the microphone 40 is transmitted to the A/D conversion circuit 410 through the wire 71 extending from the cuff 20 to the main body 10A.

The control unit 110A includes a central processing unit (CPU) as a processor, and controls the entire operation of this patient-side device 100A.

The air pipe 38a is connected to the pressure sensor 31 in a fluid flowable manner, the air pipe 38b is connected to the electric pump 32A in a fluid flowable manner, and the air pipe 38c is connected to the solenoid valve 33A in a fluid flowable manner. In this example, the air pipes 38a, 38b, and 38c merge and are connected to the cuff 20 in a fluid flowable manner as one air pipe 38.

The electric pump 32A is driven by the pump drive circuit 320 based on a control signal from the control unit 110A, and supplies air to the cuff 20 to pressurize the cuff 20. The solenoid valve 33A is opened and closed by the solenoid valve drive circuit 330 based on a control signal from the control unit 110A, and is used to discharge air from the cuff 20 or enclose air into the cuff 20. Due to this, the cuff 20 is automatically pressurized or depressurized.

The measurement button 52D is a momentary type (self-return type) switch in this example, and is configured to start a measurement action by the patient-side device 100A when depressed once, and to interrupt or stop the measurement action by the patient-side device 100A when depressed once again.

The memory 51A stores data of a program for controlling the patient-side device 100A, setting data for setting various functions of the patient-side device 100A, data during blood pressure measurement, data of a blood pressure measurement result, and the like. The memory 51A is also used as a work memory or the like when a program is executed.

The power supply unit 53A supplies or cuts off power to each unit in the main body 10A such as the control unit 110A, the memory 51, the pressure sensor 31, the electric pump 32A, the solenoid valve 33A, and other units in response to ON/OFF of the measurement button 52D.

The communication unit 190A is configured to act as a transmission unit based on a control signal from the control unit 110A during the measurement action by the patient-side device 100A. In this example, in the depressurization process of the cuff pressure Pc, the communication unit 190A sequentially transmits, as measurement data, the cuff pressure signal representing the cuff pressure Pc detected by the pressure sensor 31 alone or by temporally synchronizing the sound signal Ks output from the microphone 40 with the cuff pressure signal representing the cuff pressure Pc detected by the pressure sensor 31.

The indicator 55 incudes a liquid crystal display (LCD), and displays predetermined information in accordance with a control signal from the control unit 110A. In this example, the indicator 55 is configured to digitally display the cuff pressure Pc being measured, for example, as “○○○ mmHg”. Note that the indicator 55 may include an organic electro luminescence (EL) display or may include a light emitting diode (LED).

On the other hand, the doctor-side device 100B includes a main body 10B. The main body 10B is mounted with a control unit 110B, the indicator 50, the operation unit 52, the speaker 54 as an electro-acoustic conversion device, a memory 51B, a power supply unit 53B, and a communication unit 190B.

The control unit 110B includes a central processing unit (CPU) as a processor, and controls the entire operation of this doctor-side device 100B.

The indicator 50 and the operation unit 52 are the same as those in the sphygmomanometer 1D of FIG. 13 (therefore, the sphygmomanometer 1A of FIG. 1).

The speaker 54 reproduces the Korotkoff sound signal Kca, which is an electric signal, as the Korotkoff sound Kc based on a control signal from the control unit 110B during the measurement action by the patient-side device 100A. As the electro-acoustic conversion device, a headphone, an earphone, or the like may be provided in place of the speaker 54.

The memory 51B stores data of a program for controlling the doctor-side device 100B, setting data for setting various functions of the doctor-side device 100B, data during blood pressure measurement, data of a blood pressure measurement result, and the like. The memory 51B is also used as a work memory or the like when a program is executed.

The power supply unit 53B supplies or cuts off power to each unit in the main body 10B such as the control unit 110B, the memory 51B, and other units in response to ON/OFF of the power button 52A.

In this example, the communication unit 190B is data-communicably connected to the communication unit 190A of the patient-side device 100A via a network 190 such as the Internet. In this example, the communication unit 190B receives and passes, to the control unit 110B, the measurement data in real time from the communication unit 190A of the patient-side device 100A during the measurement action by the patient-side device 100A.

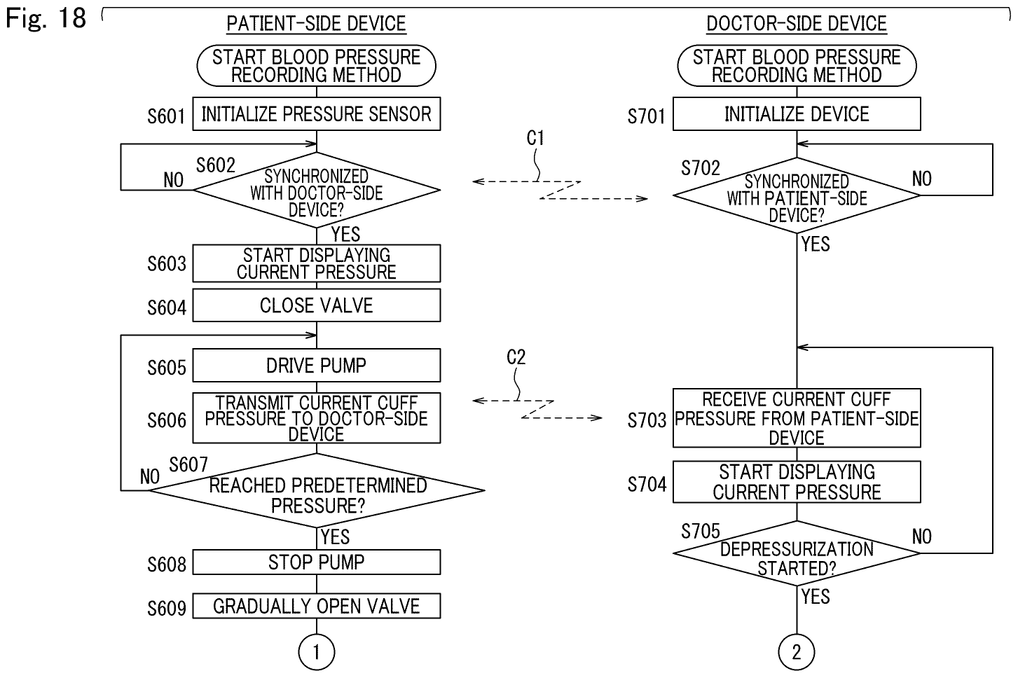

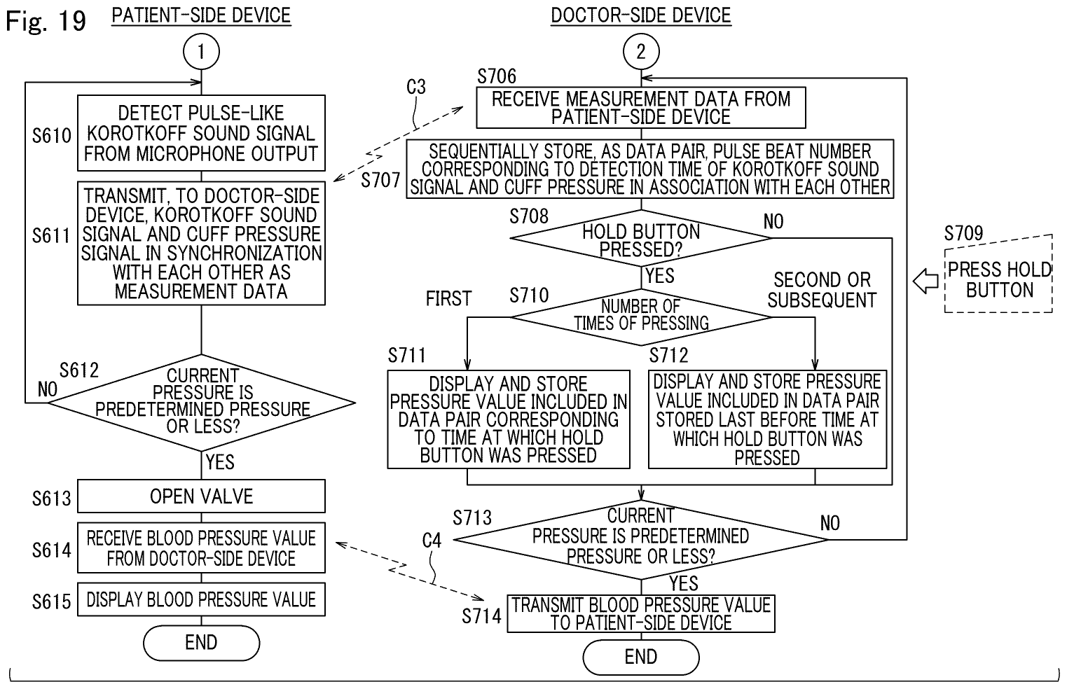

FIGS. 18 and 19 show a flow of a blood pressure recording method of an embodiment in which the observer 80 measures and records the blood pressure of the upper arm 91 of the subject 90 using the blood pressure measurement system 1F. Here, in FIGS. 18 and 19, the processing and determination performed by the control unit 110A of the patient-side device 100A are drawn with a solid line outline on the left side, and the processing and determination performed by the control unit 110B of the doctor-side device 100B are drawn with a solid line outline on the right side. Terminal 1 of the flow in FIG. 18 and terminal 1 of the flow in FIG. 19 are continuous, and terminal 2 of the flow in FIG. 18 and terminal 2 of the flow in FIG. 19 are continuous. In FIG. 19, as described above, the manual operation (i.e., manual input) by the observer 80 is drawn with a broken line outline.

It is assumed that the doctor-side device 100B is powered on in advance, and the doctor-side device 100B is initialized as shown in step S701 of FIG. 18. Accordingly, it is assumed that contents of the data set of the memory 51B are reset. As a preparation for a blood pressure measurement, this example assumes that the subject 90 wears the cuff 20 around the upper arm 91 of himself/herself.

When the subject 90 presses the measurement button 52D of the patient-side device 100A, the patient-side device 100A performs the processing of pressure sensor initialization shown in step S601 of FIG. 18.

Subsequently, as shown in steps S602 and S702 of FIG. 18, the communication unit 190A of the patient-side device 100A and the communication unit 190B of the doctor-side device 100B perform communication (this is indicated by an arrow C1 in FIG. 18) via the network 190, and synchronizes the patient-side device 100A and the doctor-side device 100B. If no synchronization is established (NO in steps S602 and S702 of FIG. 18), the process waits for a synchronization to be established. When the synchronization is established (YES in steps S602 and S702 of FIG. 18), data can be exchanged at the same timing between the patient-side device 100A and the doctor-side device 100B.

In a synchronized state, in step S603 of FIG. 18, the control unit 110A of the patient-side device 100A starts causing the indicator 55 to display the current cuff pressure Pc detected by the pressure sensor 31. Subsequently, the control unit 110A acts as a cuff pressure adjustment unit, closes the solenoid valve 33A in step S604, and starts driving of the electric pump 32A in step S605. Along with this, in step S606, the control unit 110A starts transmitting a cuff pressure signal representing the current cuff pressure Pc detected by the pressure sensor 31 from the communication unit 190A of the patient-side device 100A to the communication unit 190B of the doctor-side device 100B via the network 190 (this is indicated by an arrow C2 in FIG. 18). Furthermore, in step S607, the control unit 110A acts as the cuff pressure adjustment unit, and determines whether or not the cuff pressure Pc has reached a predetermined pressure Pcmax, which is determined in advance to be higher than an expected maximum blood pressure by about 30 mmHg as shown in the upper part of FIG. 15. If the cuff pressure Pc has not yet reached the predetermined pressure Pcmax (NO in step S607), the process returns to step S605 to continue pressurization. Due to this, the blood flow through the artery of the upper arm 91 of the subject 90 is temporarily stopped. When the cuff pressure Pc reaches the pressure Pcmax (YES in step S607 in FIG. 18), the control unit 110A stops the electric pump 32A in step S608, and gradually opens the solenoid valve 33A in step S609. Due to this, a depressurization of the cuff pressure Pc is started at a depressurization rate of typically 2 to 3 mmHg/sec. In this example, since the pressure adjustment is automatically performed in this manner, the patient as the subject 90 does not need to adjust the pressure, which is convenient.

As described above, when the cuff pressure signal representing the current cuff pressure Pc starts to be transmitted from the patient-side device 100A in step S606 of FIG. 18, the control unit 110B of the doctor-side device 100B receives in step S703 the cuff pressure signal via the communication unit 190B. Then, in step S704, the control unit 110B acts as a first display processing unit, and starts processing of causing the indicator 50 to display in real time, as the bar graph 50b along the scale 50a, the current cuff pressure Pc detected by the pressure sensor 31.

In this depressurization process of the cuff pressure Pc started in step S609 of FIG. 18, as shown in step S610 of FIG. 19, the control unit 110A of the patient-side device 100A acts as a Korotkoff sound signal detection unit, and detects a pulse-like Korotkoff sound signal Kca from a sound signal Ks output from the microphone 40 as shown in the middle part of FIG. 15. Then, in step S611 of FIG. 19, the control unit 110A temporally synchronizes the pulse-like Korotkoff sound signal Kca and the cuff pressure signal representing the cuff pressure Pc, and sequentially transmits the signals as measurement data through the communication unit 190A acting as a transmission unit (this is indicated by an arrow C3 in FIG. 19).

In step S706 of FIG. 19, the communication unit 190B of the doctor-side device 100B acts as a reception unit, receives and passes, to the control unit 110B, the measurement data in real time from the patient-side device 100A during the measurement action by the patient-side device 100A. In step S707, the control unit 110B acts as a second data accumulation unit, and causes the memory 51B to sequentially store, as a data pair, a pulse beat number N corresponding to a time at which the Korotkoff sound signal Kca was detected and the cuff pressure Pc at the time when the Korotkoff sound signal Kca was detected, in association with each other. This is set as the data set DS3 (see FIG. 15), similarly to the fourth embodiment.

In the depressurization process, the observer 80 listens to the Korotkoff sound Kc reproduced by the speaker 54 based on the Korotkoff sound signal Kca while viewing the cuff pressure Pc displayed in real time as the bar graph 50b on the indicator 50. In step S709 of FIG. 19, the observer 80 presses the HOLD button 52 at a time t1 delayed by a reaction time td1 from a time to at which he could judge that the Korotkoff sound Kc had started generating (i.e., a time at which he heard the first Korotkoff sound Kcaf from a silent state) as exemplified in the middle part of FIG. 15. If this is the first pressing in the depressurization process, the control unit 110B determines that the first timing signal t1 (for simplicity, represented by the same reference sign as the time t1) was input. Then, the control unit 110 acts as a recording processing unit, performs the processing of steps S708, S710, and S711 of FIG. 19 (same as the processing in steps S510, S512, and S513 of FIG. 14), and specifies a data pair (in the example in the lower part of FIG. 15, the data pair with the pulse beat number N=1 stored immediately before the time t1) corresponding to the time indicated by the first timing signal t1 among the data set DS3 stored in the memory 51. Furthermore, the control unit 110B causes the memory 51B to store, as a systolic blood pressure value, the cuff pressure Pc included in the specified data pair. In the example in the lower part of FIG. 15, 125 mmHg (indicated by a broken line frame C1) included in the data pair with the pulse beat number N=1 is stored as the systolic blood pressure value. Together with this, in this example, the obtained systolic blood pressure value 125 mmHg is displayed on the indicator 50.

Thereafter, the depressurization process proceeds, and the cuff pressure Pc decreases. In step S709 of FIG. 19 again, the observer 80 presses the HOLD button 52 at a time t2 delayed by a reaction time td2 from a time t21 at which he could judge that the Korotkoff sound Kc had stopped generating (i.e., a time at which he could judge for the first time that he no longer heard any Korotkoff sound after being delayed by 1 pulse beat T from a time t20 at which he could hear the last Korotkoff sound Kcae) as exemplified in the middle part of FIG. 15. If this is the second pressing in the depressurization process, the control unit 110B determines that a second timing signal t2 (for simplicity, represented by the same reference sign as the time t2) was input. Then, the control unit 110 acts as the recording processing unit, performs the processing of steps S708, S710, and S712 of FIG. 19 (same as the processing in steps S510, S512, and S514 of FIG. 14), and specifies a data pair (in the example in the lower part of FIG. 15, a data pair with the pulse beat number N=15) stored last before the time indicated by the second timing signal t2 among the data set DS3 stored in the memory 51, and causes the memory 51 to store, as a corrected diastolic blood pressure value, the cuff pressure Pc included in the specified data pair. In the example in the lower part of FIG. 15, 85 mmHg (indicated by a broken line frame C2) included in the data pair with the pulse beat number N=15 is stored as the corrected diastolic blood pressure value. Together with this, in this example, the control unit 110B acts as a third display processing unit, and causes the indicator 50 to display the corrected diastolic blood pressure value 85 mmHg. This enables the observer 80 to visually recognize the obtained systolic blood pressure value and the corrected diastolic blood pressure value.

Thereafter, when the depressurization process proceeds and the current cuff pressure Pc becomes a predetermined pressure (in this example, 30 mmHg) or less (Yes in steps S612 and S713 of FIG. 19), the control unit 110A of the patient-side device 100A opens the solenoid valve 33A in step S613. Due to this, the air is exhausted from the cuff 20 to the atmosphere through the air pipes 38 and 38c and the solenoid valve 33A. Together with this, in step S714, the control unit 110B of the doctor-side device 100B transmits the systolic blood pressure value and the corrected diastolic blood pressure value obtained above to the patient-side device 100A through the communication unit 190B (this is indicated by an arrow C4 in FIG. 19). Then, in step S614, the control unit 110A of the patient-side device 100A receives the systolic blood pressure value and the corrected diastolic blood pressure value obtained above through the communication unit 190A. In step S615, the control unit 110A of the patient-side device 100A causes the indicator 55 to display, as digital values, the systolic blood pressure value and the corrected diastolic blood pressure value obtained above. This enables the subject 90 to visually recognize the systolic blood pressure value and the corrected diastolic blood pressure value obtained above.

According to this fifth embodiment, similarly to the fourth embodiment, the systolic blood pressure value and the diastolic blood pressure value (corrected diastolic blood pressure value) can be accurately recorded. Moreover, even in a case where the location of the observer 80 (e.g., the location of the medical institution where the medical doctor as the observer 80 is working) is distant from the location of the subject 90 (e.g., the location of the residence where the patient as the subject 90 lives), the obtained systolic blood pressure value and the diastolic blood pressure value (corrected diastolic blood pressure value) can be recorded by the exchange of the measurement data between the communication unit 190A of the patient-side device 100A and the communication unit 190B of the doctor-side device 100B. The obtained systolic blood pressure value and diastolic blood pressure value (corrected diastolic blood pressure value) may be saved in an electronic medical record owned by a medical institution via, for example, the communication unit 190B.

Note that the network 190 is typically the Internet, but may be an in-hospital local area network (LAN) or one-to-one communication.

This fifth embodiment assumes that the patient-side device 100A provided in the location of the subject 90 and the doctor-side device 100B provided in the location of the observer 80 operate in synchronization. However, the present invention is not limited to this, and the patient-side device 100A and the doctor-side device 100B may operate independently. For example, after the patient-side device 100A performs the processing from step S601 of FIG. 18 to step S610 in FIG. 19 (excluding the processing in step S602 and step S606 in FIG. 18), in place of transmitting in real time the measurement data to the doctor-side device 100B in step S611 of FIG. 19, the pulse-like Korotkoff sound signal Kca and the cuff pressure signal representing the cuff pressure Pc are temporally associated with each other and temporarily recorded in the memory 51A as measurement data (this is called “second measurement data”). Thereafter, the patient-side device 100A completes the processing from steps S612 to S613 of FIG. 19. After causing the memory 51A to store the second measurement data, the patient-side device 100A transmits, at any time, to the doctor-side device 100B, the second measurement data recorded in the memory 51A in accordance with a request from the doctor-side device 100B. The doctor-side device 100B temporarily records, into the memory 51B, the received second measurement data. The observer 80 activates the doctor-side device 100B, reproduces the second measurement data temporarily recorded in the memory 51B, and decides the blood pressure value. For example, the observer 80 listens to the Korotkoff sound with the speaker 54 while viewing the cuff pressure Pc displayed on the indicator 50 based on the second measurement data, and presses the HOLD button 52B at a time when the observer 80 judges that the Korotkoff sound has started generating and/or the time at which the observer 80 judges that the Korotkoff sound has stopped generating. Due to this, the systolic blood pressure value and/or the diastolic blood pressure value (corrected diastolic blood pressure value) can be recorded based on the second measurement data. In this case, it is no longer necessary for the subject 90 and the observer 80 to simultaneously operate their respective devices. Therefore, convenience is increased for both the subject 90 and the observer 80.

In each of the above-described embodiments, the measurement target site is the upper arm 91 of the subject 90, but the present invention is not limited to this. The measurement target site may be a wrist or a lower limb of the subject 90.

In each of the above-described embodiments, the HOLD button 52B is mounted on the main body 10 of each of the sphygmomanometers 1A to 1E or the main body 100B of the doctor-side device 10, but the present invention is not limited to this. The HOLD button 52B may be provided distant from the main body 10, 10B, and may be configured to input a timing signal to the control unit 110, 110B in a wired or wireless manner.

Each of the sphygmomanometers 1A to 1E and the blood pressure measurement system 1F described above may include a printer to print out the obtained systolic blood pressure value and diastolic blood pressure value (corrected diastolic blood pressure value) on a sheet of paper such as roll paper. Each of the sphygmomanometers 1A to 1E and the blood pressure measurement system 1F described above may include a printer to print out the systolic blood pressure value and the diastolic blood pressure value (corrected diastolic blood pressure value) obtained above on a sheet of paper such as roll paper.

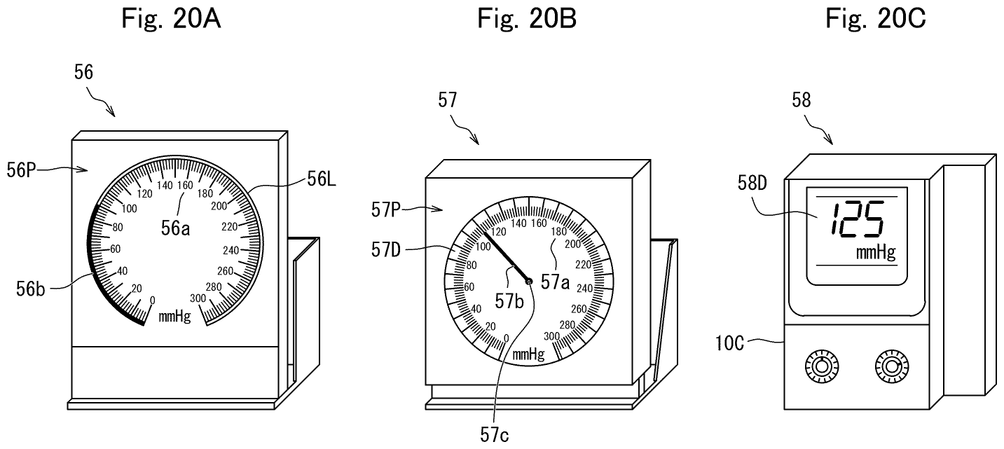

In each of the above-described embodiments, the indicator 50 for the observer 80 displays, as an image, the scale 50a arranged in the vertical direction and the bar graph 50b extending and contracting along the scale 50a together to represent the cuff pressure Pc in the form imitating a traditional mercury sphygmomanometer. However, the present invention is not limited to this. For example, indicators 56, 57, and 58 as shown in FIGS. 20A, 20B, and 20C may be adopted. The indicator 56 shown in FIG. 20A includes an LED unit 56L including a large number of LEDs arrayed in an arc shape on a front panel 56P, and a scale 56a printed in a region along the inner periphery of the LED unit 56L in the front panel 56P. The indicator 56 has a form in which the cuff pressure Pc is represented by varying a range (for convenience, the range indicated by black in FIG. 20A) 56b in which the LED unit 56L is lit. The indicator 57 shown in FIG. 20B includes an LCD unit 57D occupying a circular range provided on the front panel 56P. The indicator 57 has a form in which this LCD unit 57D displays, as an image, a scale 57a arrayed in an annular shape (or an arc shape) and a pointer 57b rotating around a center 50c as a long hand of a clock to represent the cuff pressure Pc. The indicator 58 shown in FIG. 20C includes a three-digit 7-segment (8-shaped) type LCD unit 58D provided in a main body 10C. The indicator 58 has a form in which this LCD unit 58D expresses the cuff pressure Pc as a digital value. In this manner, as the indicator for the observer 80, various forms can be used. The same applies to the indicator 55 (see FIG. 17) for the subject 90.

As described above, in a first aspect, a blood pressure recording device of the present disclosure is a blood pressure recording device configured to measure and record a blood pressure based on a Korotkoff sound generated by an artery through a measurement target site of a subject, the blood pressure recording device comprising:

a cuff worn around the measurement target site;

a cuff pressure adjustment unit for pressurizing or depressurizing a cuff pressure, the cuff pressure being a pressure of the cuff;

a pressure detection unit that detects the cuff pressure in a depressurization process of the cuff pressure by the cuff pressure adjustment unit;

a time detection unit that detects a time at which a waveform feature point of a pulse beat was generated for each pulse beat of a pulse wave indicated by the artery through the measurement target site in the depressurization process;

a first data accumulation unit that causes a memory to sequentially store, as a data pair, a time at which the waveform feature point of the pulse beat was generated or a sign corresponding to the time and the cuff pressure at the time when the waveform feature point of the pulse beat was generated, in association with each other in the depressurization process;

an indicator;

a first display processing unit that performs processing of causing the indicator to display the cuff pressure detected by the pressure detection unit; and

an operation unit configured to be operated by an observer who listens to a Korotkoff sound generated by the artery through the measurement target site with a stethoscope while viewing the cuff pressure displayed on the indicator by the first display processing unit in the depressurization process, wherein

the operation unit is configured to input a first timing signal indicating a time at which the observer judged that the Korotkoff sound had started generating and/or a second timing signal indicating a time at which the observer judged that the Korotkoff sound had stopped generating, and

the blood pressure recording device includes a recording processing unit that specifies a data pair corresponding to one pulse beat before a data pair corresponding to a time indicated by the second timing signal among a series of data pairs stored in the memory in the depressurization process, and records, as a corrected diastolic blood pressure value, the cuff pressure included in the specified data pair.

In the present description, the “subject” refers to a person whose blood pressure is to be measured, typically a patient. The “observer” refers to a person who intends to measure and record the blood pressure, typically, a medical worker such as a medical doctor or a nurse.

The “Korotkoff sound” refers to a sound generated by an artery through a measurement target site for each pulse beat in a depressurization process of cuff pressure. The “measurement target site” typically refers to an upper arm of the subject.

The “waveform feature point” of a pulse beat of a pulse wave refers to a feature point that can specify a timing in the pulse beat, such as a rising point indicating the rise of the pulse beat or a peak point indicating a peak (top of the mountain) of the pulse beat.

The “sign corresponding to the time” at which the waveform feature point was generated indicates a serial number (pulse beat number) assigned to each pulse beat, for example.

The “operation unit” widely includes means to allow an observer to input a timing signal, such as a switch that is temporarily turned on at a timing when pressed such as a HOLD button in known examples, or a touch key that is temporarily turned on at a timing when touched.

The “data pair corresponding to a time indicated by . . . timing signal” refers to a data pair stored immediately before (specifically, immediately before by a reaction time of the observer) the time indicated by the timing signal among a series of data pairs stored in the memory in the depressurization process.

In the blood pressure recording device of this first aspect, the cuff is worn around the measurement target site of the subject as preparation for blood pressure measurement. A stethoscope is brought into contact with a part (typically, a part downstream of the cuff) through which the artery of the measurement target site passes. In this state, after the cuff pressure, which is a pressure of the cuff, is pressurized by the cuff pressure adjustment unit and the blood flow in the artery through the measurement target site is temporarily stopped, the cuff pressure is detected by the pressure detection unit in the depressurization process of the cuff pressure. Together with this, in the depressurization process, the time detection unit detects the time at which the waveform feature point of the pulse wave is generated for each pulse beat of a pulse wave indicated by the artery through the measurement target site. In the depressurization process, the first data accumulation unit causes the memory to sequentially stores, as a data pair, a time at which the waveform feature point of the pulse beat was generated or a sign corresponding to the time and the cuff pressure at the time when the waveform feature point of the pulse beat was generated, in association with each other. Furthermore, in the depressurization process, an observer, who listens to a Korotkoff sound generated by the artery through the measurement target site with the stethoscope while viewing the cuff pressure displayed on the indicator by the first display processing unit, inputs at least a second timing signal indicating a time at which he/she judged that the Korotkoff sound had stopped generating by operating the operation unit. Then, the recording processing unit specifies a data pair corresponding to one pulse beat before a data pair corresponding to the time indicated by the second timing signal among a series of data pairs stored in the memory in the depressurization process, and records, as a corrected diastolic blood pressure value, the cuff pressure included in the specified data pair.

In this manner, in the blood pressure recording device of this first aspect, the recording processing unit specifies a data pair corresponding to one pulse beat before a data pair corresponding to the time indicated by the second timing signal among a series of data pairs stored in the memory in the depressurization process, and records, as a corrected diastolic blood pressure value, the cuff pressure included in the specified data pair. According to the analysis of the present inventor described above, the corrected diastolic blood pressure value represents not the time at which the observer can judge for the first time that he/she no longer hears the Korotkoff sound from a state where he/she continued to hear the Korotkoff sound for each pulse beat but the cuff pressure corresponding to the time at which he/she actually heard the last Korotkoff sound.

Therefore, according to this blood pressure recording device, it is possible to correct a phenomenon that the minimum blood pressure is evaluated lower by about one pulse beat equivalent.

As described in Non-Patent Document 2, it is considered that a slight reaction time is required from when the observer judged that the Korotkoff sound had stopped generating to when he/she inputs the second timing signal by operating the operation unit. However, in the blood pressure recording device of this first aspect, in the depressurization process, the first data accumulation unit sequentially stores, in the memory, the data pair (i.e., the time at which the waveform feature point of the pulse beat was generated or a sign corresponding to the time, and the cuff pressure at the time when the waveform feature point of the pulse beat was generated) without being affected by such a reaction time. Therefore, the data pair specified by the recording processing unit is stored without being affected by the reaction time of the observer. It is assumed that the reaction time of the observer normally does not exceed one pulse beat. As a result, the corrected diastolic blood pressure value is not actually affected by the reaction time of the observer.

Note that for each of the pulse beats, there may be a slight deviation (time interval) between the time at which the waveform feature point of the pulse beat is generated and the time at which the Korotkoff sound of the pulse beat is generated. However, normally, the deviation does not reach half a pulse beat. Therefore, the deviation does not substantially reduce the accuracy of blood pressure measurement (recording).

As described above, according to the blood pressure recording device of this first aspect, the diastolic blood pressure value can be accurately recorded as compared with known examples.

In the blood pressure recording device of one embodiment,

the recording processing unit specifies a data pair corresponding to a time indicated by the first timing signal among the series of data pairs stored in the memory, and records, as a systolic blood pressure value, the cuff pressure included in the specified data pair.

According to the analysis of the present inventor described above, the time indicated by the first timing signal corresponds to the time at which the observer judged that the Korotkoff sound had started generating, that is, the time at which the observer judged that he/she heard the first Korotkoff sound from a silent state. As described in Non-Patent Document 2, it is considered that a slight reaction time is required from when the observer judged that the Korotkoff sound had started generating to when he/she inputs the first timing signal by operating the operation unit. However, as described above, the data pair specified by the recording processing unit is stored without being affected by the reaction time of the observer. It is assumed that the reaction time of the observer normally does not exceed one pulse beat. As a result, in the blood pressure recording device of this one embodiment, the recorded systolic blood pressure value is not actually affected by the reaction time of the observer. Therefore, according to the blood pressure recording device of this one embodiment, the systolic blood pressure value can be accurately recorded.

In the blood pressure recording device of one embodiment,

the recording processing unit determines whether an operation of the operation unit is a first operation or a second or subsequent operation during the depressurization process, records the systolic blood pressure value using, as the time indicated by the first timing signal, a time at which the first operation was performed, and records the corrected diastolic blood pressure value using, as the time indicated by the second timing signal, a time at which the second or subsequent operation was performed.

In a typical case, the observer is assumed to intend to record the systolic blood pressure value when operating the operation unit for the first time and record the diastolic blood pressure value (in the present invention, the corrected diastolic blood pressure value) when operating the operation unit for the second time. Therefore, in the blood pressure recording device of this one embodiment, the recording processing unit determines whether an operation of the operation unit is a first operation or a second or subsequent operation during the depressurization process, records the systolic blood pressure value using, as the time indicated by the first timing signal, a time at which the first operation was performed. The recording processing unit records the corrected diastolic blood pressure value using, as the time indicated by the second timing signal, a time at which the second or subsequent operation was performed. Thus, according to the blood pressure recording device of this one embodiment, both the systolic blood pressure value and the corrected diastolic blood pressure value can be quickly recorded. The observer can end the blood pressure measurement as soon as the corrected diastolic blood pressure value can be recorded even in the middle of the depressurization process.

Note that second “or subsequent” is intended to be able to cope with the possibility that the operation unit is operated three times or more during the depressurization process in a case where the subject exhibits arrhythmias, a case where the subject is pregnant, or the like.

In the blood pressure recording device of one embodiment,

the operation unit includes a first switch for receiving an instruction for recording a systolic blood pressure value and a second switch for receiving an instruction for recording a diastolic blood pressure value, and

during the depressurization process, the recording processing unit records the systolic blood pressure value using, as the time indicated by the first timing signal, a time at which the first switch was operated, and records the corrected diastolic blood pressure value using, as the time indicated by the second timing signal, a time at which the second switch was operated.

In the blood pressure recording device of this one embodiment, during the depressurization process, the observer operates the first switch in order to record the systolic blood pressure value at the time when the observer judges that the Korotkoff sound has started generating. Then, the recording processing unit records the systolic blood pressure value using the time at which the first switch was operated as the time indicated by the first timing signal during the depressurization process. During the depressurization process, the observer operates the second switch in order to record the diastolic blood pressure value at the time when the observer judges that the Korotkoff sound has stopped generating. Then, the recording processing unit records the corrected diastolic blood pressure value using the time at which the second switch was operated as the time indicated by the second timing signal during the depressurization process. Thus, according to the blood pressure recording device of this one embodiment, both the systolic blood pressure value and the corrected diastolic blood pressure value can be quickly recorded according to the intention of the observer (whether to record the systolic blood pressure value or to record the diastolic blood pressure value). The observer can end the blood pressure measurement as soon as the corrected diastolic blood pressure value can be recorded even in the middle of the depressurization process. Moreover, since the recording processing unit does not need to determine whether the operation of the operation unit is the first operation or the second operation in the depressurization process, the processing by the recording processing unit is simplified.

The blood pressure recording device of one embodiment further comprises:

a data pair specification unit that, every time the operation unit is operated during the depressurization process, specifies a data pair corresponding to a time at which the operation unit was operated, and maintains information indicating a specified data pair;

a second display processing unit that causes the indicator to display, as an option, the cuff pressure included in the data pair specified by the data pair specification unit from among the series of data pairs stored in the memory in the depressurization process; and

a selection operation unit configured for selecting an option that, the observer considers, falls under the diastolic blood pressure value among the options displayed on the indicator, wherein

after the depressurization process ending, the recording processing unit records the corrected diastolic blood pressure value by using, as the data pair corresponding to the time indicated by the second timing signal, a data pair including the cuff pressure represented by the option selected by the selection operation unit among the series of data pairs stored in the memory in the depressurization process.

In the present description, “a data pair corresponding to a time at which the operation unit was operated” refers to a data pair stored immediately before (specifically, immediately before by the reaction time of the observer) the time at which the operation unit was operated among a series of data pairs stored in the memory in the depressurization process.

The “after the depressurization process ending” refers to, for example, after the cuff pressure reaches a predetermined pressure (e.g., 30 mmHg) at which the diastolic blood pressure is not normally assumed.

The “selection operation unit” widely includes means that can select an option (e.g., a mark representing a pressure value by a position along a scale) displayed on the indicator.

In the blood pressure recording device of this one embodiment, the data pair specification unit specifies a data pair corresponding to a time at which the operation unit is operated every time the operation unit is operated during the depressurization process, and maintains information indicating each specified data pair. The second display processing unit causes the indicator to display, as an option (e.g., a mark indicating a pressure value by a position along the scale), the cuff pressure included in the data pair specified by the data pair specification unit from among the series of data pairs stored in the memory in the depressurization process. The observer selects an option that, the observer considers, corresponds to the diastolic blood pressure among the options displayed on the indicator with the selection operation unit. Then, after the depressurization process ending, the recording processing unit records the corrected diastolic blood pressure value by using, as the data pair corresponding to the time indicated by the second timing signal, a data pair including the cuff pressure represented by the option selected by the selection operation unit among the series of data pairs stored in the memory in the depressurization process. Thus, according to the blood pressure recording device of this one embodiment, the corrected diastolic blood pressure value can be recorded according to the selection of the observer (selection of which option corresponding to the diastolic blood pressure).

The blood pressure recording device of one embodiment further comprises:

a data pair specification unit that, every time the operation unit is operated during the depressurization process, specifies a data pair corresponding to a time at which the operation unit was operated, and maintains information indicating a specified data pair, wherein

after the depressurization process ending, the recording processing unit records the corrected diastolic blood pressure value by using, as the data pair corresponding to the time indicated by the second timing signal, a data pair corresponding to a time at which a last operation was performed among data pairs specified by the data pair specification unit.

In the blood pressure recording device of this one embodiment, during the depressurization process, the data pair specification unit specifies a data pair corresponding to a time at which the operation unit is operated every time the operation unit is operated, and maintains information indicating each specified data pair. After the depressurization process ending, the recording processing unit records the corrected diastolic blood pressure value by using, as the data pair corresponding to the time indicated by the second timing signal, a data pair corresponding to a time at which a last operation was performed among data pairs specified by the data pair specification unit. According to the blood pressure recording device of this one embodiment, the processing by the recording processing unit is simplified.

The blood pressure recording device of one embodiment further comprises:

a third display processing unit that causes the indicator to display the corrected diastolic blood pressure value as a digital value and/or as a mark indicating a pressure value by a position along a scale.

In the blood pressure recording device of this one embodiment, the third display processing unit causes the indicator to display the corrected diastolic blood pressure value as a digital value and/or as a mark indicating a pressure value by a position along a scale. Therefore, the observer can easily recognize the corrected diastolic blood pressure value by viewing the indicator.

In the blood pressure recording device of one embodiment,

the first data accumulation unit is configured to start to cause the memory to sequentially store the data pair from a data pair corresponding to a time at which the first operation was performed by the operation unit during the depressurization process.

In the blood pressure recording device of this one embodiment, the resource of the memory can be saved as compared with a case where the first data accumulation unit starts causing the memory to sequentially store the data pair simultaneously with a start of the depressurization process, for example.

In a second aspect, a blood pressure recording method of the present disclosure is a blood pressure recording method for measuring and recording a blood pressure of a measurement target site of a subject using the blood pressure recording device according to claim 1, the blood pressure recording method comprising:

wearing the cuff around the measurement target site, and bringing the stethoscope into contact with a part through which the artery of the measurement target site passes;

after pressurizing the cuff pressure by the cuff pressure adjustment unit to temporarily stop a blood flow in the artery through the measurement target site, in the depressurization process of the cuff pressure,

detecting the cuff pressure by the pressure detection unit;

detecting, by time detection unit, a signal representing the time at which the waveform feature point of the pulse beat was generated for each pulse beat of the pulse wave indicated by the artery through the measurement target site;

causing, by the first data accumulation unit, the memory to sequentially store, as the data pair, the time at which the waveform feature point of the pulse beat was generated or the sign corresponding to the time and the cuff pressure at the time when the waveform feature point of the pulse beat was generated, in association with each other;

inputting the second timing signal indicating at least the time at which the observer judged that the Korotkoff sound had stopped generating, by operating the operation unit by the observer who listens to the Korotkoff sound generated by the artery through the measurement target site with the stethoscope while viewing the cuff pressure displayed on the indicator by the first display processing; and

during the depressurization process or after the depressurization process ending, by the recording processing unit, specifying the data pair corresponding to one pulse beat before the data pair corresponding to the time indicated by the second timing signal among the series of data pairs stored in the memory in the depressurization process, and recording, as the corrected diastolic blood pressure value, the cuff pressure included in the specified data pair.

According to the blood pressure recording method of this second aspect, the diastolic blood pressure value can be accurately recorded as compared with known examples.

In a third aspect, a blood pressure recording device of the present disclosure is a blood pressure recording device configured to measure and record a blood pressure based on a Korotkoff sound generated by an artery through a measurement target site of a subject, the blood pressure recording device comprising:

a cuff worn around the measurement target site;

a cuff pressure adjustment unit for pressurizing or depressurizing a cuff pressure, the cuff pressure being a pressure of the cuff;

a pressure detection unit that detects the cuff pressure in a depressurization process of the cuff pressure by the cuff pressure adjustment unit;

a microphone that converts a sound generated by the artery through the measurement target site into a sound signal that is an electric signal and outputs the sound signal in the depressurization process;

a Korotkoff sound signal detection unit that detects a Korotkoff sound signal indicating a Korotkoff sound from the sound signal output by the microphone;

a second data accumulation unit that causes a memory to sequentially store, as a data pair, a time at which the Korotkoff sound signal was detected or a sign corresponding to the time and the cuff pressure at the time when the Korotkoff sound signal was detected, in association with each other in the depressurization process;

an indicator;

a first display processing unit that performs processing of causing the indicator to display the cuff pressure detected by the pressure detection unit; and

an operation unit configured to be operated by an observer who listens to the Korotkoff sound generated by the artery through the measurement target site with a stethoscope or listens to the Korotkoff sound indicated by the Korotkoff sound signal with an electro-acoustic conversion device while viewing the cuff pressure displayed on the indicator by the first display processing unit in the depressurization process, wherein

the operation unit is configured to input a first timing signal indicating a time at which the observer judged that the Korotkoff sound had started generating and/or a second timing signal indicating a time at which the observer judged that the Korotkoff sound had stopped generating, and

the blood pressure recording device includes a recording processing unit that specifies a data pair stored last before the time indicated by the second timing signal among a series of data pairs stored in the memory in the depressurization process, and records, as a corrected diastolic blood pressure value, the cuff pressure included in the specified data pair.

In the present description, the “Korotkoff sound signal” refers to a pulse-like electric signal indicating a Korotkoff sound. The “electro-acoustic conversion device” refers to a device that converts an electric signal into sound, for example, a speaker, a headphone, an earphone, or the like.

In the blood pressure recording device of this third aspect, the cuff is worn around the measurement target site of the subject as preparation for blood pressure measurement. The microphone is arranged so as to acquire sound generated by an artery through the measurement target site. Alternatively, in addition to the arrangement of the microphone, a stethoscope is brought into contact with a part (typically, a part downstream of the cuff) through which the artery of the measurement target site passes. In this state, after the cuff pressure, which is a pressure of the cuff, is pressurized by the cuff pressure adjustment unit and the blood flow in the artery through the measurement target site is temporarily stopped, the cuff pressure is detected by the pressure detection unit in the depressurization process of the cuff pressure. Together with this, in the depressurization process, the Korotkoff sound detection unit detects a Korotkoff sound signal indicating a Korotkoff sound from the sound signal output from the microphone. Alternatively, in addition to that, the microphone converts a sound generated by the artery through the measurement target site into a sound signal that is an electric signal and outputs the sound signal. In the depressurization process, the second data accumulation unit causes the memory to sequentially stores, as a data pair, a time at which the Korotkoff sound signal was detected or a sign corresponding to the time and the cuff pressure at the time when the Korotkoff sound signal was detected, in association with each other. Furthermore, in the depressurization process, an observer, who listens to the Korotkoff sound generated by the artery through the measurement target site with a stethoscope or listens to the Korotkoff sound indicated by the Korotkoff sound signal with an electro-acoustic conversion device while viewing the cuff pressure displayed on the indicator by the first display processing unit in the depressurization process, inputs at least a second timing signal indicating a time at which he/she judged that the Korotkoff sound had stopped generating by operating the operation unit. Then, the recording processing unit specifies a data pair stored last before the time indicated by the second timing signal among a series of data pairs stored in the memory in the depressurization process, and records, as a corrected diastolic blood pressure value, the cuff pressure included in the specified data pair.

In this manner, in the blood pressure recording device of this third aspect, the recording processing unit specifies a data pair stored last before the time indicated by the second timing signal among a series of data pairs stored in the memory in the depressurization process, and records, as a corrected diastolic blood pressure value, the cuff pressure included in the specified data pair. According to the analysis of the present inventor described above, the corrected diastolic blood pressure value represents not the time at which the observer can judge for the first time that he/she no longer hears the Korotkoff sound from a state where he/she continued to hear the Korotkoff sound for each pulse beat but the cuff pressure corresponding to the time at which he/she actually heard the last Korotkoff sound. Therefore, according to this blood pressure recording device, it is possible to correct a phenomenon that the minimum blood pressure is evaluated lower by about one pulse beat equivalent.

As described in Non-Patent Document 2 (George S. Stergiou et al, “Validation of the A&D UM-101 professional hybrid device for office blood pressure measurement according to the International Protocol”, Blood Pressure Monitoring 2008, Vol. 13, No. 1, pp 37-42), it is considered that a slight reaction time is required from when the observer judged that the Korotkoff sound had stopped generating to when he/she inputs the second timing signal by operating the operation unit. However, in the blood pressure recording device of this third aspect, in the depressurization process, the second data accumulation unit sequentially stores, in the memory, the data pair (i.e., the time at which the Korotkoff sound signal was detected or a sign corresponding to the time, and the cuff pressure at the time when the Korotkoff sound signal was detected) without being affected by such a reaction time. Therefore, the data pair specified by the recording processing unit is stored without being affected by the reaction time of the observer. As a result, the corrected diastolic blood pressure value is not actually affected by the reaction time of the observer.

As described above, according to the blood pressure recording device of this third aspect, the diastolic blood pressure value can be accurately recorded as compared with known examples.

In the blood pressure recording device of one embodiment,

in a location of the subject having the measurement target site,

the cuff, the cuff pressure adjustment unit, the pressure detection unit, the microphone, and the Korotkoff sound signal detection unit are arranged,

the location is provided with a transmission unit that temporally synchronizes and sequentially transmits, as measurement data, the Korotkoff sound signal detected by the Korotkoff sound signal detection unit and a cuff pressure signal representing the cuff pressure detected by the pressure detection unit in the depressurization process,

in a location of the observer distant from the location of the subject,

a reception unit that receives the measurement data is provided, and

the second data accumulation unit, the memory, the indicator, the electro-acoustic conversion device, the first display processing unit, the operation unit, and the recording processing unit are arranged,

based on the measurement data received in the depressurization process,

the first display processing unit causes the indicator to display the cuff pressure, and

the second data accumulation unit causes the memory to sequentially store, as the data pair, the time at which the Korotkoff sound signal was detected or a sign corresponding to the time and the cuff pressure at the time when the Korotkoff sound signal was detected, in association with each other, and

in the depressurization process, the observer, who listens to the Korotkoff sound indicated by the Korotkoff sound signal with the electro-acoustic conversion device while viewing the cuff pressure displayed on the indicator by the first display processing unit, inputs at least the second timing signal by operating the operation unit.

In the present description, the term “location of a subject” typically refers to a location of a residence where a patient as a subject lives. The “location of an observer” typically refers to a location of a medical institution where a medical doctor or a nurse as an observer is working.

In the blood pressure recording device of this one embodiment, the cuff is worn around the measurement target site of the subject at the location of the subject. The microphone is arranged so as to acquire sound generated by the artery through the measurement target site. In this state, after the cuff pressure is pressurized by the cuff pressure adjustment unit and the blood flow in the artery through the measurement target site is temporarily stopped, the cuff pressure is detected by the pressure detection unit in the depressurization process of the cuff pressure. Together with this, in the depressurization process, the transmission unit temporally synchronizes and sequentially transmits, as measurement data, the sound signal output from the microphone and a cuff pressure signal representing the cuff pressure detected by the pressure detection unit.

On the other hand, the measurement data is received by the reception unit at the location of the observer distant from the location of the subject. In the depressurization process, the first display processing unit displays the cuff pressure on the indicator based on the received measurement data. In synchronization with display of the cuff pressure, the Korotkoff sound signal detection unit detects the Korotkoff sound signal from the sound signal included in the measurement data. The second data accumulation unit causes the memory to sequentially stores, as a data pair, a time at which the Korotkoff sound signal was detected or a sign corresponding to the time and the cuff pressure at the time when the Korotkoff sound signal was detected, in association with each other. Furthermore, in the depressurization process, the observer, who listens to the Korotkoff sound indicated by the Korotkoff sound signal with the electro-acoustic conversion device while viewing the cuff pressure displayed on the indicator by the first display processing unit, inputs at least the second timing signal by operating the operation unit. Then, the recording processing unit specifies a data pair stored last before the time indicated by the second timing signal among a series of data pairs stored in the memory in the depressurization process, and records, as a corrected diastolic blood pressure value, the cuff pressure included in the specified data pair.

According to the blood pressure recording device of this one embodiment, the diastolic blood pressure value can be accurately recorded as compared with known examples. Moreover, even in a case where the location of the observer (e.g., the location of the medical institution where the medical doctor or the nurse as the observer is working) is distant from the location of the subject (e.g., the location of the residence where the patient as the subject lives), the corrected diastolic blood pressure value can be recorded by exchange of the measurement data between the transmission unit and the reception unit.

In a fourth aspect, a blood pressure recording method of the present disclosure is a blood pressure recording method for measuring and recording a blood pressure of a measurement target site of a subject using the blood pressure recording device according to claim 10, the blood pressure recording method comprising:

wearing the cuff around the measurement target site;

arranging the microphone so as to acquire the sound generated by the artery through the measurement target site, or in addition to arrangement of the microphone, bringing a stethoscope into contact with a part through which the artery of the measurement target site passes;

after pressurizing the cuff pressure by the cuff pressure adjustment unit to temporarily stop a blood flow in the artery through the measurement target site, in the depressurization process of the cuff pressure,

detecting the cuff pressure by the pressure detection unit;

converting, by the microphone, the sound generated by the artery through the measurement target site into the sound signal and outputting the sound signal;

detecting, by the Korotkoff sound signal detection unit, the Korotkoff sound signal from the sound signal output by the microphone;

causing, by the second data accumulation unit, the memory to sequentially store, as the data pair, the time at which the Korotkoff sound signal was detected or the sign corresponding to the time and the cuff pressure at the time when the Korotkoff sound signal was detected, in association with each other;

inputting the second timing signal indicating at least the time at which the observer judged that the Korotkoff sound had stopped generating, by operating the operation unit by the observer who listens to the Korotkoff sound generated by the artery through the measurement target site with the stethoscope or listens to the Korotkoff sound indicated by the Korotkoff sound signal with the electro-acoustic conversion device while viewing the cuff pressure displayed on the indicator by the first display processing; and

during the depressurization process or after the depressurization process ending, by the recording processing unit, specifying the data pair stored last before the time indicated by the second timing signal among the series of data pairs stored in the memory in the depressurization process, and recording, as the corrected diastolic blood pressure value, the cuff pressure included in the specified data pair.

According to the blood pressure recording method of this fourth aspect, the diastolic blood pressure value can be accurately recorded as compared with known examples.

In the blood pressure recording device of one embodiment,

the first display processing unit is configured to perform processing of causing the indicator to display, as a bar graph along a scale, the cuff pressure detected by the pressure detection unit.

According to the blood pressure recording device of this one embodiment, the observer can judge that the Korotkoff sound has started generating and/or that the Korotkoff sound has stopped generating, with the same sense as viewing a traditional mercury sphygmomanometer. Therefore, it can be said that it is convenient for an observer who is accustomed to using a traditional mercury sphygmomanometer.

As clear from the above, according to the blood pressure recording device and the blood pressure recording method of the present disclosure, it is possible to accurately record a diastolic blood pressure value (minimum blood pressure) when an observer operates an operation unit (e.g., such as the above HOLD button) based on the Korotkoff sound.

The above embodiments are illustrative, and are modifiable in a variety of ways without departing from the scope of this invention. It is to be noted that the various embodiments described above can be appreciated individually within each embodiment, but the embodiments can be combined together. It is also to be noted that the various features in different embodiments can be appreciated individually by its own, but the features in different embodiments can be combined.

1. A blood pressure recording device configured to measure and record a blood pressure based on a Korotkoff sound generated by an artery through a measurement target site of a subject, the blood pressure recording device comprising:
a cuff worn around the measurement target site;
a cuff pressure adjustment unit for pressurizing or depressurizing a cuff pressure, the cuff pressure being a pressure of the cuff;
a pressure detection unit that detects the cuff pressure in a depressurization process of the cuff pressure by the cuff pressure adjustment unit;
a time detection unit that detects a time at which a waveform feature point of a pulse beat was generated for each pulse beat of a pulse wave indicated by the artery through the measurement target site in the depressurization process;
a first data accumulation unit that causes a memory to sequentially store, as a data pair, a time at which the waveform feature point of the pulse beat was generated or a sign corresponding to the time and the cuff pressure at the time when the waveform feature point of the pulse beat was generated, in association with each other in the depressurization process;
an indicator;
a first display processing unit that performs processing of causing the indicator to display the cuff pressure detected by the pressure detection unit; and
an operation unit configured to be operated by an observer who listens to a Korotkoff sound generated by the artery through the measurement target site with a stethoscope while viewing the cuff pressure displayed on the indicator by the first display processing unit in the depressurization process, wherein
the operation unit is configured to input a first timing signal indicating a time at which the observer judged that the Korotkoff sound had started generating and/or a second timing signal indicating a time at which the observer judged that the Korotkoff sound had stopped generating, and
the blood pressure recording device includes a recording processing unit that specifies a data pair corresponding to one pulse beat before a data pair corresponding to a time indicated by the second timing signal among a series of data pairs stored in the memory in the depressurization process, and records, as a corrected diastolic blood pressure value, the cuff pressure included in the specified data pair.

2. The blood pressure recording device according to claim 1, wherein
the recording processing unit specifies a data pair corresponding to a time indicated by the first timing signal among the series of data pairs stored in the memory, and records, as a systolic blood pressure value, the cuff pressure included in the specified data pair.

3. The blood pressure recording device according to claim 2, wherein
the recording processing unit determines whether an operation of the operation unit is a first operation or a second or subsequent operation during the depressurization process, records the systolic blood pressure value using, as the time indicated by the first timing signal, a time at which the first operation was performed, and records the corrected diastolic blood pressure value using, as the time indicated by the second timing signal, a time at which the second or subsequent operation was performed.

4. The blood pressure recording device according to claim 2, wherein
the operation unit includes a first switch for receiving an instruction for recording a systolic blood pressure value and a second switch for receiving an instruction for recording a diastolic blood pressure value, and
during the depressurization process, the recording processing unit records the systolic blood pressure value using, as the time indicated by the first timing signal, a time at which the first switch was operated, and records the corrected diastolic blood pressure value using, as the time indicated by the second timing signal, a time at which the second switch was operated.

5. The blood pressure recording device according to claim 1, further comprising:
a data pair specification unit that, every time the operation unit is operated during the depressurization process, specifies a data pair corresponding to a time at which the operation unit was operated, and maintains information indicating a specified data pair;
a second display processing unit that causes the indicator to display, as an option, the cuff pressure included in the data pair specified by the data pair specification unit from among the series of data pairs stored in the memory in the depressurization process; and
a selection operation unit configured for selecting an option that, the observer considers, falls under the diastolic blood pressure value among the options displayed on the indicator, wherein
after the depressurization process ending, the recording processing unit records the corrected diastolic blood pressure value by using, as the data pair corresponding to the time indicated by the second timing signal, a data pair including the cuff pressure represented by the option selected by the selection operation unit among the series of data pairs stored in the memory in the depressurization process.

6. The blood pressure recording device according to claim 1, further comprising:
a data pair specification unit that, every time the operation unit is operated during the depressurization process, specifies a data pair corresponding to a time at which the operation unit was operated, and maintains information indicating a specified data pair, wherein
after the depressurization process ending, the recording processing unit records the corrected diastolic blood pressure value by using, as the data pair corresponding to the time indicated by the second timing signal, a data pair corresponding to a time at which a last operation was performed among data pairs specified by the data pair specification unit.

7. The blood pressure recording device according to claim 1, further comprising:
a third display processing unit that causes the indicator to display the corrected diastolic blood pressure value as a digital value and/or as a mark indicating a pressure value by a position along a scale.

8. The blood pressure recording device according to claim 1, wherein
the first data accumulation unit is configured to start to cause the memory to sequentially store the data pair from a data pair corresponding to a time at which the first operation was performed by the operation unit during the depressurization process.

9. A blood pressure recording method for measuring and recording a blood pressure of a measurement target site of a subject using the blood pressure recording device according to claim 1, the blood pressure recording method comprising:
wearing the cuff around the measurement target site, and bringing the stethoscope into contact with a part through which the artery of the measurement target site passes;
after pressurizing the cuff pressure by the cuff pressure adjustment unit to temporarily stop a blood flow in the artery through the measurement target site, in the depressurization process of the cuff pressure,
detecting the cuff pressure by the pressure detection unit;
detecting, by time detection unit, a signal representing the time at which the waveform feature point of the pulse beat was generated for each pulse beat of the pulse wave indicated by the artery through the measurement target site;
causing, by the first data accumulation unit, the memory to sequentially store, as the data pair, the time at which the waveform feature point of the pulse beat was generated or the sign corresponding to the time and the cuff pressure at the time when the waveform feature point of the pulse beat was generated, in association with each other;
inputting the second timing signal indicating at least the time at which the observer judged that the Korotkoff sound had stopped generating, by operating the operation unit by the observer who listens to the Korotkoff sound generated by the artery through the measurement target site with the stethoscope while viewing the cuff pressure displayed on the indicator by the first display processing; and
during the depressurization process or after the depressurization process ending, by the recording processing unit, specifying the data pair corresponding to one pulse beat before the data pair corresponding to the time indicated by the second timing signal among the series of data pairs stored in the memory in the depressurization process, and recording, as the corrected diastolic blood pressure value, the cuff pressure included in the specified data pair.

10. A blood pressure recording device configured to measure and record a blood pressure based on a Korotkoff sound generated by an artery through a measurement target site of a subject, the blood pressure recording device comprising:
a cuff worn around the measurement target site;
a cuff pressure adjustment unit for pressurizing or depressurizing a cuff pressure, the cuff pressure being a pressure of the cuff;
a pressure detection unit that detects the cuff pressure in a depressurization process of the cuff pressure by the cuff pressure adjustment unit;
a microphone that converts a sound generated by the artery through the measurement target site into a sound signal that is an electric signal and outputs the sound signal in the depressurization process;
a Korotkoff sound signal detection unit that detects a Korotkoff sound signal indicating a Korotkoff sound from the sound signal output by the microphone;
a second data accumulation unit that causes a memory to sequentially store, as a data pair, a time at which the Korotkoff sound signal was detected or a sign corresponding to the time and the cuff pressure at the time when the Korotkoff sound signal was detected, in association with each other in the depressurization process;
an indicator;
a first display processing unit that performs processing of causing the indicator to display the cuff pressure detected by the pressure detection unit; and
an operation unit configured to be operated by an observer who listens to the Korotkoff sound generated by the artery through the measurement target site with a stethoscope or listens to the Korotkoff sound indicated by the Korotkoff sound signal with an electro-acoustic conversion device while viewing the cuff pressure displayed on the indicator by the first display processing unit in the depressurization process, wherein
the operation unit is configured to input a first timing signal indicating a time at which the observer judged that the Korotkoff sound had started generating and/or a second timing signal indicating a time at which the observer judged that the Korotkoff sound had stopped generating, and
the blood pressure recording device includes a recording processing unit that specifies a data pair stored last before the time indicated by the second timing signal among a series of data pairs stored in the memory in the depressurization process, and records, as a corrected diastolic blood pressure value, the cuff pressure included in the specified data pair.

11. The blood pressure recording device according to claim 10, wherein
in a location of the subject having the measurement target site,
the cuff, the cuff pressure adjustment unit, the pressure detection unit, the microphone, and the Korotkoff sound signal detection unit are arranged,
the location is provided with a transmission unit that temporally synchronizes and sequentially transmits, as measurement data, the Korotkoff sound signal detected by the Korotkoff sound signal detection unit and a cuff pressure signal representing the cuff pressure detected by the pressure detection unit in the depressurization process,
in a location of the observer distant from the location of the subject,
a reception unit that receives the measurement data is provided, and
the second data accumulation unit, the memory, the indicator, the electro-acoustic conversion device, the first display processing unit, the operation unit, and the recording processing unit are arranged,
based on the measurement data received in the depressurization process,
the first display processing unit causes the indicator to display the cuff pressure, and
the second data accumulation unit causes the memory to sequentially store, as the data pair, the time at which the Korotkoff sound signal was detected or a sign corresponding to the time and the cuff pressure at the time when the Korotkoff sound signal was detected, in association with each other, and
in the depressurization process, the observer, who listens to the Korotkoff sound indicated by the Korotkoff sound signal with the electro-acoustic conversion device while viewing the cuff pressure displayed on the indicator by the first display processing unit, inputs at least the second timing signal by operating the operation unit.

12. A blood pressure recording method for measuring and recording a blood pressure of a measurement target site of a subject using the blood pressure recording device according to claim 10, the blood pressure recording method comprising:
wearing the cuff around the measurement target site;
arranging the microphone so as to acquire the sound generated by the artery through the measurement target site, or in addition to arrangement of the microphone, bringing a stethoscope into contact with a part through which the artery of the measurement target site passes;
after pressurizing the cuff pressure by the cuff pressure adjustment unit to temporarily stop a blood flow in the artery through the measurement target site, in the depressurization process of the cuff pressure,
detecting the cuff pressure by the pressure detection unit;
converting, by the microphone, the sound generated by the artery through the measurement target site into the sound signal and outputting the sound signal;
detecting, by the Korotkoff sound signal detection unit, the Korotkoff sound signal from the sound signal output by the microphone;
causing, by the second data accumulation unit, the memory to sequentially store, as the data pair, the time at which the Korotkoff sound signal was detected or the sign corresponding to the time and the cuff pressure at the time when the Korotkoff sound signal was detected, in association with each other;
inputting the second timing signal indicating at least the time at which the observer judged that the Korotkoff sound had stopped generating, by operating the operation unit by the observer who listens to the Korotkoff sound generated by the artery through the measurement target site with the stethoscope or listens to the Korotkoff sound indicated by the Korotkoff sound signal with the electro-acoustic conversion device while viewing the cuff pressure displayed on the indicator by the first display processing; and
during the depressurization process or after the depressurization process ending, by the recording processing unit, specifying the data pair stored last before the time indicated by the second timing signal among the series of data pairs stored in the memory in the depressurization process, and recording, as the corrected diastolic blood pressure value, the cuff pressure included in the specified data pair.

13. The blood pressure recording device according to claim 1, wherein
the first display processing unit is configured to perform processing of causing the indicator to display, as a bar graph along a scale, the cuff pressure detected by the pressure detection unit.

14. The blood pressure recording device according to claim 10, wherein
the first display processing unit is configured to perform processing of causing the indicator to display, as a bar graph along a scale, the cuff pressure detected by the pressure detection unit.
